(19) United States
(12) Patent Application Publication
(10) Pub. No.: US 2019/0151232 A1
(43) Pub. Date: May 23, 2019

(54) Topical Compositions, Process of Manufacture and Method of Use

(71) Applicant: David SCHARP, Aliso Viejo, CA (US)

(72) Inventor: David SCHARP, Aliso Viejo, CA (US)

(73) Assignee: Progeneron, Aliso Viejo, CA (US)

(21) Appl. No.: 16/198,793

(22) Filed: Nov. 22, 2018

Related U.S. Application Data
(60) Provisional application No. 62/590,251, filed on Nov. 22, 2017.

(51) Int. Cl. A61K 9/00; A61K 31/78; A61K 31/015; A61K 36/185; A61K 47/20; A61K 31/047; A61K 36/71; A61K 36/886; A61K 38/39; A61K 31/685; A61K 47/08; A61K 47/44; A61K 47/46; A61K 31/192; A61K 31/245; A61K 47/26; A61K 31/355; A61K 31/197; A61K 31/4172; A61K 31/085; B01F 3/08; B01F 7/00; B01F 15/06

(52) U.S. Cl. CPC A61K 9/0014; B01F 2215/0034; A61K 31/015; A61K 36/185; A61K 47/20; A61K 31/047; A61K 36/71; A61K 36/886; A61K 38/39; A61K 31/685; A61K 47/08; A61K 47/44; A61K 47/46; A61K 31/192; A61K 31/245; A61K 47/26; A61K 31/355; A61K 31/197; A61K 31/4172; A61K 31/085; B01F 3/0853; B01F 7/00341; B01F 15/06; B01F 2015/061; B01F 2015/062; A61K 31/78

(57) ABSTRACT

Compositions for the treatment of some orphan diseases and oral mucosal ulcers, many with similarities in terms of their anti-inflammatory and anti-oxidative activities, but also multiple differences in their observed abilities that can be combined to challenge the current, underlying pathophysiology. The orphan diseases of interest are Dupuytren's Contracture, Peyronie's Disease, Scleroderma, Raynaud's (or Renaud's) Phenomenon, chemotherapy/radiation induced oral mucosal ulceration, and aphthous ulcers; and more frequent skin issues of skin damage from cuts, abrasions, and burns; aging skin changes, and toe nail fungus. These can be treated with the disclosed compositions with the proper combination and alteration of ingredients:

### CROSS-REFERENCE TO RELATED APPLICATIONS

This is a Non-Provisional application, which claims priority to U.S. Provisional Application No. 62/590,251, which was filed on Nov. 22, 2017; the contents of which are all herein incorporated by this reference in their entireties. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### BACKGROUND OF INVENTION

### Field of the Invention

The application discloses compositions for treatment of topical orphan diseases and related syndromes, and methods of treatment and process of manufacture of disclosed compositions.

### Description of the Related Art

Treatments for orphan diseases poses problems not associated with more frequent diseases. First, since few people are inflected, there is little awareness of their problems, so few treatments are investigated. Additionally the low frequency of occurrence hinders the development of these treatments due to the feeling that it will be difficult for a company to recover the high costs of development.

An orphan disease is defined as a condition that affects fewer than 200,000 people nationwide. Collectively, however, they affect as many as 25 million Americans, according to the National Institutes of Health (NIH), and that makes the diseases—and finding treatments for them—a serious public health concern.

Orphan diseases include several well-known diseases, like cystic fibrosis and Tourette's syndrome, but most are unknown to the general public. The orphan diseases of interest in this application are Dupuytren's Contracture, Peyronie's Disease, Scleroderma, Raynaud's (or Renaud's) Phenomenon, Chemotherapy/Radiation Induced Oral Mucosal Ulceration, and Aphthous Ulcers; and more frequent skin issues of Skin Damage from Cuts, Abrasions, and Burns; Aging Skin Changes, and Toe Nail Fungus.

Patients diagnosed with a rare disease are often denied access to effective medicines because prescription drug manufacturers rarely could make a profit from marketing drugs to such small groups. Consequently, the prescription drug industry has not adequately funded research for orphan product development. Despite the urgent health need for these medicines, they came to be known as orphans because companies were not interested in adopting them.

Dupuytren's Contracture is one of the diseases with few people but who suffer great pain and difficulty with life. About 15 million Americans age 35 and older have Dupuytren disease, which is about 5% of the US population. 3 million in the US have bent fingers from Dupuytren disease and 750,000 Americans have severe Dupuytren biology and the risk of being crippled even with available treatment. After age 55, the percent of people with Dupuytren disease increases steadily in both men and women. By age 70, one-quarter of men have signs of Dupuytren, increasing to one-third by age 80. Dupuytren is more common in women than men in those 85 and older. The total number of US Dupuytren cases increased from 14.2 million in 2000 to 16.2 million people in 2010. The percent of the US population 35 and older affected with Dupuytren disease increased from 8.7% in 2000 to 9.3% in 2010.

Dupuytren's Contracture is a rare connective tissue disorder characterized by fixation of the joints of certain fingers in a permanently flexed position. Due to abnormal thickening and shortening of the bands of fibrous tissue beneath the skin of the palm, a hardened nodule may develop, eventually forming an abnormal band of hardened fibrotic tissue. This causes the fingers of the affected area to begin to be drawn in toward the palm over several months or years and cannot be pulled back. The skin of the affected area may pucker. The ring and pinky fingers are most affected and usually both hands are affected.

Peyronie disease is similar to Dupuytren's Contracture as both involve a connective tissue disorder. Peyronie disease is characterized by the development of fibrous plaques in the soft tissue of the penis of adult males which results in penile deformity. It is estimated to affect 0.5% adult males in the United States, although it is difficult to know the actual extent due to the hesitancy of disclosing the problem. Affected individuals may experience pain, have cord-like lesions on the penis, and exhibit abnormal curvature of the penis when erect. The abnormal curvature of the penis may make it impossible for affected individuals to have normal sexual intercourse.

Scleroderma is a rare, chronic disease of the immune system, blood vessels and connective tissue. 2.5 million people worldwide have scleroderma with an estimated 300,000 people in the United States. The disease is three to four times more common in females than in males. Scleroderma may occur at any age but the symptoms most frequently begin during midlife. The symptoms of scleroderma are similar to those of other autoimmune diseases so diagnosis is difficult.

Scleroderma is an autoimmune connective tissue disorder characterized by abnormal thickening of the skin. Connective tissue is composed of collagen, which supports and binds other body tissues. Some types of scleroderma affect certain, specific parts of the body, while other types can affect the whole body and internal organs. The exact cause of scleroderma is unknown.

The early symptoms of scleroderma vary considerably but distinctive abnormalities on the skin usually appear later in the course of the disease. Common symptoms include painful joints, morning stiffness, fatigue, and weight loss. People with scleroderma have areas of skin that become hard and leathery. These areas of hardness are widespread and typically appear on both sides of the body. Eventually, tissue loss occurs and the skin becomes more highly colored. The skin can become thin, shiny, and bright which results in decreased function of the fingers and toes.

Treatment of scleroderma is usually just symptomatic and supportive. Medications can be given to control the hardening of the skin, although not very effective, and skin care may include lubricating creams or antibiotic ointments for infected ulcerations.

Another orphan disease, Raynaud's phenomenon, can be associated with scleroderma, although it can occur in people without scleroderma. Raynaud's phenomenon is a vascular disorder characterized by the intermittent loss of blood to various parts of the body, particularly the fingers, toes, nose, and ears causing them to turn white. This typically occurs after exposure to cold and causes tingling sensations, numbness, and pain. Raynaud's phenomenon is an early complaint of people with scleroderma. Drug therapy with Vasodilators may help widen the blood vessels.

An estimated 28 million people in the US have Raynaud's phenomenon. The major symptom is a dramatic stark white pallor of the affected areas when exposed to cold, although a blue or red color may also be present from time to time. Other symptoms include a feeling of numbness, severe aching or pain, tingling or throbbing, a sensation of tightness, “pins and needles,” and a profound loss of sensation.

Another condition that causes severe pain and difficulty is oral mucosal ulceration induced chemotherapy/radiation. Although it is not a disease itself, oral mucosal ulceration is a result of treatments for cancer. Radiation-induced oral mucosal ulceration occurs in up to 80% of head and neck cancer irradiated patients and reaches up to 100% in patients with altered fractionation head and neck cancer.

Radiation-induced oral mucosal ulceration is a major dose-limiting toxicity in head and neck cancer patients. It is a normal tissue injury caused by radiation, which has marked adverse effects on patient quality of life and cancer therapy continuity. It is a challenge for proper treatment since it leads to cancer therapy interruption, poor local tumor control, and changes in dose fractionation. Its economic cost is estimated to be about 17,000 dollars per patient with head and neck cancers.

Aphthous Ulcers (also called canker sores) are similar to the oral mucosal ulceration caused by cancer treatments, but occur in people without a known cause. Aphthous ulcers are small, shallow lesions that develop on the soft tissues in the mouth or at the base of the gums. Aphthous ulcers do not occur on the surface of the lips and they are not contagious. However, they are painful and can make eating difficult. Most sores disappear on their own in a week or two, but some can last longer.

The aphthous ulcers are round or oval with a white or yellow center and a red border. They form inside the mouth; on or under the tongue; inside the cheeks or lips, at the base of the gums; or on the soft palate. There is also a tingling or burning sensation a day or two before the sores appear.

Another problem with treating these orphan diseases was the inability to furnish compositions with sufficient concentrations of active ingredients. Additionally, some of the active ingredients were hydrophilic and other active ingredients were hydrophobic. It is difficult to apply hydrophilic and hydrophobic ingredients at the same time in sufficient concentrations as to be effective against the disease.

Previously, the hydrophilic and hydrophobic ingredients would separate before application, which prevented their combined use. The investigator of this application was able to solve this formulation problem that had plagued other researchers. Additionally, the disclosed process of manufacture presented unexpected results in both ingredient concentration and compatibility.

Additionally, it was discovered during the development of the disclosed compositions and treatments that the compositions were additionally effective against more common skin and oral problems. Variations of the compositions for the orphan diseases were able to treat skin damage from cuts, abrasions, and burns; aging skin changes; and toe nail fungus. This discovery increased the population who could be helped by these compositions.

The disclosed compositions and methods relate to treating medical conditions that are known to have few, if any, effective treatments currently available. There are many medical conditions that exist today without currently demonstrated effective medical treatments that are the subject of extensive research by major pharmaceutical companies throughout the world. The goal is to develop potentially beneficial, skin care compositions that demonstrate new effectiveness following composition component alteration and manufacturing in multiple medical conditions without known and effective treatments.

### BRIEF SUMMARY OF THE INVENTION

Tables 1 and 2 provide the percentage of hydrophobic and hydrophilic components in a disclosed composition.

TABLE 1

| Hydrophobic | | Component % of |
|---|---|---|
| Components | Function | Composition |
| α-Pinene (APN) | anti-inflammatory, anti- | 0.00% to 50.00% |
| | microbial | |
| Acai Berry | anti-inflammatory | 0.00% to 50.00% |
| Anethole Anise Camphor | anti-microbial | 0.00% to 50.00% |
| Apricot Kernal Oil | functional oil | 0.00% to 50.00% |
| (AKO) | | |
| Argan Nut Oil (AGN) | skin disorders | 0.00% to 50.00% |
| Baobab Oil (BAB) | moisturizer | 0.00% to 50.00% |
| Bees Wax (BW) | phase change emulsion | 0.00% to 50.00% |
| Benzoin (BZN) | thickener | 0.00% to 50.00% |
| Benzyl Alcohol (BA) | solvent, low toxicity | 0.00% to 50.00% |
| Black Cumin Seed | oil | 0.00% to 50.00% |
| (Nigella sativa) | | |
| Cabreuva Oil | anti-microbial | 0.00% to 50.00% |
| Caffeic acid | anti-oxidant | 0.00% to 50.00% |
| Calendula Oil (CDA) | anti-inflammatory | 0.00% to 50.00% |
| Camellia Oil (CMA) | anti-oxidant | 0.00% to 50.00% |
| Capaiba Oil (CPO) | anti-inflammatory | 0.00% to 50.00% |
| Caprylic, Capric | soft skin | 0.00% to 50.00% |
| Triglyceride (CCT) | | |
| Carvacrol (CVC) | anti-microbial | 0.00% to 50.00% |
| Carvone Oil (CRV) | fragrance | 0.00% to 50.00% |
| Caryophyllene (CAP) | anti-oxidant | 0.00% to 50.00% |
| Cedarwood essential Oil | flavor | 0.00% to 50.00% |
| Cetyl Alcohol (CTA) | alcohol | 0.00% to 50.00% |
| Chamomile German | fragrance | 0.00% to 50.00% |
| Oil (CGB) | | |
| Elemi Oil (EMO) | functional oil | 0.00% to 50.00% |
| Geranium Oil (GER) | essential oil | 0.00% to 50.00% |
| Ghana Shea Butter | salve, moisturize | 0.00% to 50.00% |
| (GSB) | | |
| Glycerol (GYO) | moisturizer, solvent | 0.00% to 50.00% |
| Ho Wood Oil (HWO) | fragrance | 0.00% to 50.00% |
| Jasmine Oil (JAS) | essential oil | 0.00% to 50.00% |
| Kaempferol | anti-oxidant | 0.00% to 50.00% |
| Kanuka Oil (KNO) | functional oil | 0.00% to 50.00% |
| Lavandin Oil (LVO) | fragrance | 0.00% to 50.00% |
| Limonene Oil (LMN) | fragrance | 0.00% to 50.00% |
| Litsea Cubeba Oil (LCO) | functional oil | 0.00% to 50.00% |
| Marula Oil (MRL) | moisture, anti-oxidant | 0.00% to 50.00% |
| Myrrh | anti-inflammatory | 0.00% to 50.00% |
| Nerolina Oil (NRL) | fragrance | 0.00% to 50.00% |
| Niaouli Oil | anti-microbial | 0.00% to 50.00% |
| Palmarosa Oil (PLO) | traditional, anti-microbial | 0.00% to 50.00% |
| Palo Santo | functional oil | 0.00% to 50.00% |
| Panthenol (PNT) | alcohol | 0.00% to 50.00% |
| Peppermint | fragrance | 0.00% to 50.00% |
| Rose Hip Oil (RHO) | healing, anti-inflammatory | 0.00% to 50.00% |
| Rosemary Oil (RMO) | floral | 0.00% to 50.00% |
| Sacha Inchi Oil (SIO) | supportive skin care | 0.00% to 50.00% |
| Sea Buckthorn Oil (SBO) | repair, anti-oxidant | 0.00% to 50.00% |
| Tamanu Oil (TMU) | skin care | 0.00% to 50.00% |
| Terpineol | fragrance | 0.00% to 50.00% |
| Thyme Red, White | anti-microbial | 0.00% to 50.00% |
| Vetiver | fragrance | 0.00% to 50.00% |
| Vitamin E (VTE) | cell reactions, anti-oxidant | 0.00% to 50.00% |
| Winter Savory | anti-microbial | 0.00% to 50.00% |

TABLE 2

| | | Component % of |
|---|---|---|
| Hydrophilic Components | Function | Composition |
| 1-Tetradecanol | moisturizer | 0.00% to 50.00% |
| Agave (AGV) | cell nutrients, fructose, | 0.00% to 50.00% |
| | sucrose | |
| Alanine (ALA) | amino acid for collagen | 0.00% to 50.00% |
| Aloe Vera Gel (AVG) | skin healing, anti- | 0.00% to 50.00% |
| | inflammatory | |
| Arginine (ARG) | amino acid | 0.00% to 50.00% |
| Artic Fish Collagen (AFC) | critical soluble protein | 0.00% to 50.00% |
| Benzocaine (BZC) | topical analgesic | 0.00% to 50.00% |
| Distilled water | solvent | 0.00% to 50.00% |
| Ferulic Acid (FRA) | anti-oxidant | 0.00% to 50.00% |
| Glutamine (GLU) | amino acid | 0.00% to 50.00% |
| Glycine (GLY) | amino acid for collagen | 0.00% to 50.00% |
| Histidine (HST) | amino acid | 0.00% to 50.00% |
| Hydroxyproline (HDP) | amino acid | 0.00% to 50.00% |
| Isoleucine (ISL) | amino acid | 0.00% to 50.00% |
| Lecithin (LCT) | skin soft and smooth | 0.00% to 50.00% |
| Leucine (LUC) | amino acid | 0.00% to 50.00% |
| Linoleic Acid (LOA) | anti-inflammatory | 0.00% to 50.00% |
| Lysine (LYS) | amino acid | 0.00% to 50.00% |
| Methyl sulfonyl methane | transport | 0.00% to 50.00% |
| (MSM) | | |
| Niacinamide (NCM) | vitamin b3, no flush | 0.00% to 50.00% |
| Oleic Acid (OLA) | anti-inflammatory | 0.00% to 50.00% |
| Proline (PRL) | amino acid for collagen | 0.00% to 50.00% |
| Protocatechuic Acid (PCT) | anti-oxidant | 0.00% to 50.00% |
| Sericin | gel | 0.00% to 50.00% |
| Sodium Polyacrylate | moisturizer | 0.00% to 50.00% |
| Vitamin A (VTA) | vitamin | 0.00% to 50.00% |
| Vitamin B3 (VTB3) or | vitamin b3 skin health, | 0.00% to 50.00% |
| Niacin | vasodilate | |
| Vitamin C (VTC) | tissue repair, anti-oxidant | 0.00% to 50.00% |

The meaning of the stated range of 0.00% to 50.00% is that every value from 0.00 to 50.00 to the 0.01 place is disclosed and any range combining any two values in the range is possible. Thus, each whole number 0, 1, 2, 3 . . . 46, 47, 48, 49, 50 is disclosed. Additionally, every 0.1 unit between each whole number is disclosed. Therefore, between each whole number the values of 0.1, 0.2, 0.3 . . . 0.6, 0.7, 0.8, and 0.9 are disclosed. Additionally, every 0.01 unit between each 0.1 is disclosed. Therefore, between each 0.1 unit the values of 0.01, 0.02, 0.03 . . . 0.06, 0.07, 0.08, 0.09 are disclosed.

The stated range of 0.00% to 50.00% includes any range within this range. This range includes 5,000 values—0.00, 0.001 . . . 49.98, 49.99, 50.00. Therefore, the 12,502,500 different ranges possible by stating a minimum value and maximum value are disclosed by the stated range of 0.00% to 50.00%.

The temperatures in the method can be modified depending on the component being combined and the final format of the composition. The temperature can be given as less than or equal to a value. The temperature can be given as greater than or equal to a value. The temperatures can be given as a range between any two disclosed values.

In one embodiment the composition can be a lotion, solution, balm, cream, spray or other consistency used for topical application.

In one embodiment the composition can be applied topically or oral mucosal application.

In one embodiment the composition can be a pharmaceutical, medicinal, cosmetic, or over-the-counter.

In one embodiment the amount of a component is in milliliters.

In one embodiment the amount of a component is in grams.

In one embodiment the amount of a component is in percent of total.

In one embodiment the amount of α-Pinene is from 30 ml to 90 ml per batch of composition. In another embodiment the amount of α-Pinene is from 50 ml to 70 ml per batch of composition. In another embodiment the amount of α-Pinene is 60 ml per batch of composition.

In one embodiment the amount of 1-Tetradecanol is from 1.0 g to 5.0 g per batch of composition. In another embodiment the amount of 1-Tetradecanol is from 1.5 g to 3.5 g per batch of composition. In another embodiment the amount of 1-Tetradecanol is 2.5 g per batch of composition.

In one embodiment the amount of Acai Berry is from 1 ml to 20 ml per batch of composition. In another embodiment the amount of Acai Berry is from 5 ml to 15 ml per batch of composition. In another embodiment the amount of Acai Berry is 10 ml per batch of composition.

In one embodiment the amount of Artic Fish Collagen is from 10 g to 70 g per batch of composition. In another embodiment the amount of Artic Fish Collagen is from 30 g to 50 g per batch of composition. In another embodiment the amount of Artic Fish Collagen is 40 g per batch of composition.

In one embodiment the amount of Agave nectar is from 1 ml to 20 ml per batch of composition. In another embodiment the amount of Agave nectar is from 5 ml to 15 ml per batch of composition. In another embodiment the amount of Agave nectar is 10 ml per batch of composition.

In one embodiment the amount of Alanine is from 1 g to 10 g per batch of composition. In another embodiment the amount of Alanine is from 2.5 g to 7.5 g per batch of composition. In another embodiment the amount of Alanine is 5 g per batch of composition.

In one embodiment the amount of Aloe Vera Leaf Gel is from 100 g to 500 g per batch of composition. In another embodiment the amount of Aloe Vera Leaf Gel is from 200 g to 350 g per batch of composition. In another embodiment the amount of Aloe Vera Leaf Gel is 275 g per batch of composition.

In one embodiment the amount of Anethole Anise Camphor is from 1 ml to 10 ml per batch of composition. In another embodiment the amount of Anethole Anise Camphor is from 2.5 ml to 7.5 ml per batch of composition. In another embodiment the amount of Anethole Anise Camphor is 5 ml per batch of composition.

In one embodiment the amount of Apricot Kernal Oil is from 1 ml to 10 ml per batch of composition. In another embodiment the amount of Apricot Kernal Oil is from 2.5 ml to 7.5 ml per batch of composition. In another embodiment the amount of Apricot Kernal Oil is 5 ml per batch of composition.

In one embodiment the amount of Argan Nut Oil is from 0.5 ml to 10 ml per batch of composition. In another embodiment the amount of Argan Nut Oil is from 1.0 ml to 7.5 ml per batch of composition. In another embodiment the amount of Argan Nut Oil is 2.5-5.0 ml per batch of composition.

In one embodiment the amount of Arginine is from 1 g to 10 g per batch of composition. In another embodiment the amount of Arginine is from 2.5 g to 7.5 g per batch of composition. In another embodiment the amount of Arginine is 5 g per batch of composition.

In one embodiment the amount of Baobab Oil is from 0.5 ml to 10 ml per batch of composition. In another embodiment the amount of Baobab Oil is from 1.0 ml to 7.5 ml per batch of composition. In another embodiment the amount of Baobab Oil is 2.5-5.0 ml per batch of composition.

In one embodiment the amount of Bees Wax is from 10 g to 70 g per batch of composition. In another embodiment the amount of Bees Wax is from 30 g to 50 g per batch of composition. In another embodiment the amount of Bees Wax is 40 g per batch of composition.

In one embodiment the amount of Benzocaine is from 1 g to 7 g per batch of composition. In another embodiment the amount of Benzocaine is from 3 g to 5 g per batch of composition. In another embodiment the amount of Benzocaine is 4 g per batch of composition.

In one embodiment the amount of Benzoin is from 1 ml to 7 ml per batch of composition. In another embodiment the amount of Benzoin is from 3 ml to 5 ml per batch of composition. In another embodiment the amount of Benzoin is 4 ml per batch of composition.

In one embodiment the amount of Benzyl Alcohol is from 1 ml to 100 ml per batch of composition. In another embodiment the amount of Benzyl Alcohol is from 25 ml to 75 ml per batch of composition. In another embodiment the amount of Benzyl Alcohol is 50 ml per batch of composition.

In one embodiment the amount of Cabreuva Oil is from 1 ml to 10 ml per batch of composition. In another embodiment the amount of Cabreuva Oil is from 2.5 ml to 7.5 ml per batch of composition. In another embodiment the amount of Cabreuva Oil is 5 ml per batch of composition.

In one embodiment the amount of Caffeic acid is from 1 g to 10 g per batch of composition. In another embodiment the amount of Caffeic acid is from 2.5 g to 7.5 g per batch of composition. In another embodiment the amount of Caffeic acid is 5 g per batch of composition.

In one embodiment the amount of Calendula Oil is from 1 ml to 10 ml per batch of composition. In another embodiment the amount of Calendula Oil is from 2.5 ml to 7.5 ml per batch of composition. In another embodiment the amount of Calendula Oil is 5 ml per batch of composition.

In one embodiment the amount of Camellia Oil is from 1 ml to 20 ml per batch of composition. In another embodiment the amount of Camellia Oil is from 5 ml to 15 ml per batch of composition. In another embodiment the amount of Camellia Oil is 10 ml per batch of composition.

In one embodiment the amount of Capaiba Oil is from 1 ml to 20 ml per batch of composition. In another embodiment the amount of Capaiba Oil is from 5 ml to 15 ml per batch of composition. In another embodiment the amount of Capaiba Oil is 10 ml per batch of composition.

In one embodiment the amount of Caprylic, Capric Triglyceride is from 1 ml to 20 ml per batch of composition. In another embodiment the amount of Caprylic, Capric Triglyceride is from 5 ml to 15 ml per batch of composition. In another embodiment the amount of Caprylic, Capric Triglyceride is 10 ml per batch of composition.

In one embodiment the amount of Carvacrol is from 1 ml to 10 ml per batch of composition. In another embodiment the amount of Carvacrol is from 2.5 ml to 7.5 ml per batch of composition. In another embodiment the amount of Carvacrol is 5 ml per batch of composition.

In one embodiment the amount of Carvone Oil is from 1.0 ml to 5.0 ml per batch of composition. In another embodiment the amount of Carvone Oil is from 1.5 ml to 3.5 ml per batch of composition. In another embodiment the amount of Carvone Oil is 2.5 ml per batch of composition.

In one embodiment the amount of Caryophyllene is from 1 ml to 20 ml per batch of composition. In another embodiment the amount of Caryophyllene is from 5 ml to 15 ml per batch of composition. In another embodiment the amount of Caryophyllene is 10 ml per batch of composition.

In one embodiment the amount of Cedarwood Oil is from 1.0 ml to 5.0 ml per batch of composition. In another embodiment the amount of Cedarwood Oil is from 1.5 ml to 3.5 ml per batch of composition. In another embodiment the amount of Cedarwood Oil is 2.5 ml per batch of composition.

In one embodiment the amount of Cetyl Alcohol is from 10 g to 70 g per batch of composition. In another embodiment the amount of Cetyl Alcohol is from 30 g to 50 g per batch of composition. In another embodiment the amount of Cetyl Alcohol is 40 g per batch of composition.

In one embodiment the amount of Chamomile German Oil is from 1 ml to 20 ml per batch of composition. In another embodiment the amount of Chamomile German Oil is from 5 ml to 15 ml per batch of composition. In another embodiment the amount of Chamomile German Oil is 10 ml per batch of composition.

In one embodiment the amount of Distilled water is from 100 ml to 1,000 ml per batch of composition. In another embodiment the amount of Distilled water is from 250 ml to 750 ml per batch of composition. In another embodiment the amount of Distilled water is 500 ml per batch of composition.

In one embodiment the amount of Elemi Oil is from 1 ml to 10 ml per batch of composition. In another embodiment the amount of Elemi Oil is from 2.5 ml to 7.5 ml per batch of composition. In another embodiment the amount of Elemi Oil is 5 ml per batch of composition.

In one embodiment the amount of Ferulic Acid is from 1 g to 20 g per batch of composition. In another embodiment the amount of Ferulic Acid is from 5 g to 15 g per batch of composition. In another embodiment the amount of Ferulic Acid is 10 g per batch of composition.

In one embodiment the amount of Geranium Oil is from 1.0 ml to 5.0 ml per batch of composition. In another embodiment the amount of Geranium Oil is from 1.5 ml to 3.5 ml per batch of composition. In another embodiment the amount of Geranium Oil is 2.5 ml per batch of composition.

In one embodiment the amount of Ghana Shea Butter is from 100 g to 500 g per batch of composition. In another embodiment the amount of Ghana Shea Butter is from 200 g to 350 g per batch of composition. In another embodiment the amount of Ghana Shea Butter is 275 g per batch of composition.

In one embodiment the amount of Glutamine is from 1 g to 10 g per batch of composition. In another embodiment the amount of Glutamine is from 2.5 g to 7.5 g per batch of composition. In another embodiment the amount of Glutamine is 5 g per batch of composition.

In one embodiment the amount of Glycerol is from 1 ml to 20 ml per batch of composition. In another embodiment the amount of Glycerol is from 5 ml to 15 ml per batch of composition. In another embodiment the amount of Glycerol is 10 ml per batch of composition.

In one embodiment the amount of Glycine is from 1 g to 10 g per batch of composition. In another embodiment the amount of Glycine is from 2.5 g to 7.5 g per batch of composition. In another embodiment the amount of Glycine is 5 g per batch of composition.

In one embodiment the amount of Histidine is from 1 g to 10 g per batch of composition. In another embodiment the amount of Histidine is from 2.5 g to 7.5 g per batch of composition. In another embodiment the amount of Histidine is 5 g per batch of composition.

In one embodiment the amount of Ho Wood Oil is from 1.0 ml to 5.0 ml per batch of composition. In another embodiment the amount of Ho Wood Oil is from 1.5 ml to 3.5 ml per batch of composition. In another embodiment the amount of Ho Wood Oil is 2.5 ml per batch of composition.

In one embodiment the amount of Hydroxyproline is from 1 g to 10 g per batch of composition. In another embodiment the amount of Hydroxyproline is from 2.5 g to 7.5 g per batch of composition. In another embodiment the amount of Hydroxyproline is 5 g per batch of composition.

In one embodiment the amount of Isoleucine is from 1 g to 10 g per batch of composition. In another embodiment the amount of Isoleucine is from 2.5 g to 7.5 g per batch of composition. In another embodiment the amount of Isoleucine is 5 g per batch of composition.

In one embodiment the amount of Jasmine Oil is from 1.0 ml to 5.0 ml per batch of composition. In another embodiment the amount of Jasmine Oil is from 1.5 ml to 3.5 ml per batch of composition. In another embodiment the amount of Jasmine Oil is 2.5 ml per batch of composition.

In one embodiment the amount of Kaempferol is from 1 g to 10 g per batch of composition. In another embodiment the amount of Kaempferol is from 2.5 g to 7.5 g per batch of composition. In another embodiment the amount of Kaempferol is 5 g per batch of composition.

In one embodiment the amount of Kanuka Oil is from 1.0 ml to 5.0 ml per batch of composition. In another embodiment the amount of Kanuka Oil is from 1.5 ml to 3.5 ml per batch of composition. In another embodiment the amount of Kanuka Oil is 2.5 ml per batch of composition.

In one embodiment the amount of Lavodin Oil is from 1 ml to 20 ml per batch of composition. In another embodiment the amount of Lavodin Oil is from 5 ml to 15 ml per batch of composition. In another embodiment the amount of Lavodin Oil is 10 ml per batch of composition.

In one embodiment the amount of Lecithin is from 10 g to 50 g per batch of composition. In another embodiment the amount of Lecithin is from 15 g to 35 g per batch of composition. In another embodiment the amount of Lecithin is 25 g per batch of composition.

In one embodiment the amount of Leucine is from 1 g to 10 g per batch of composition. In another embodiment the amount of Leucine is from 2.5 g to 7.5 g per batch of composition. In another embodiment the amount of Leucine is 5 g per batch of composition.

In one embodiment the amount of Limonene Oil is from 100 ml to 1,000 ml per batch of composition. In another embodiment the amount of Limonene Oil is from 200 ml to 600 ml per batch of composition. In another embodiment the amount of Limonene Oil is 400 ml per batch of composition.

In one embodiment the amount of Linoleic Acid is from 1 ml to 10 ml per batch of composition. In another embodiment the amount of Linoleic Acid is from 2.5 ml to 7.5 ml per batch of composition. In another embodiment the amount of Linoleic Acid is 5 ml per batch of composition.

In one embodiment the amount of Litsea Cubebe Oil is from 1 ml to 10 ml. In another embodiment the amount of Litsea Cubebe Oil is from 2.5 ml to 7.5 ml per batch of composition. In another embodiment the amount of Litsea Cubebe Oil is 5 ml per batch of composition.

In one embodiment the amount of Lysine is from 1 g to 10 g per batch of composition. In another embodiment the amount of Lysine is from 2.5 g to 7.5 g per batch of composition. In another embodiment the amount of Lysine is 5 g per batch of composition.

In one embodiment the amount of Marula Oil is from 1.0 ml to 5.0 ml per batch of composition. In another embodiment the amount of Marula Oil is from 1.5 ml to 3.5 ml per batch of composition. In another embodiment the amount of Marula Oil is 2.5 ml per batch of composition.

In one embodiment the amount of Methyl Sulfonyl Methane is from 10 g to 50 g per batch of composition. In another embodiment the amount of Methyl Sulfonyl Methane is from 10 g to 50 g per batch of composition. In another embodiment the amount of Methyl Sulfonyl Methane is 25 g per batch of composition.

In one embodiment the amount of Myrrh is from 1 ml to 10 ml per batch of composition. In another embodiment the amount of Myrrh is from 2.5 ml to 7.5 ml per batch of composition. In another embodiment the amount of Myrrh is 5 ml per batch of composition.

In one embodiment the amount of Nerolina Oil is from 1.0 ml to 5.0 ml per batch of composition. In another embodiment the amount of Nerolina Oil is from 1.5 ml to 3.5 ml per batch of composition. In another embodiment the amount of Nerolina Oil is 2.5 ml per batch of composition.

In one embodiment the amount of Niacinamide is from 1.0 g to 5.0 g per batch of composition. In another embodiment the amount of Niacinamide is from 1.5 g to 3.5 g per batch of composition. In another embodiment the amount of Niacinamide is 2.5 g per batch of composition.

In one embodiment the amount of Niaouli Oil is from 1 ml to 10 ml per batch of composition. In another embodiment the amount of Niaouli Oil is from 2.5 ml to 7.5 ml per batch of composition. In another embodiment the amount of Niaouli Oil is 5 ml per batch of composition.

In one embodiment the amount of Nigella saliva Black Cumin Seed Oil is from 10 ml to 100 ml per batch of composition. In another embodiment the amount of Nigella saliva Black Cumin Seed Oil is from 25 ml to 75 ml per batch of composition. In another embodiment the amount of Nigella saliva Black Cumin Seed Oil is 50 ml per batch of composition.

In one embodiment the amount of Oleic Acid is from 1 ml to 10 ml per batch of composition. In another embodiment the amount of Oleic Acid is from 2.5 ml to 7.5 ml per batch of composition. In another embodiment the amount of Oleic Acid is 5 ml per batch of composition.

In one embodiment the amount of Palmarosa Oil is from 1.0 ml to 5.0 ml per batch of composition. In another embodiment the amount of Palmarosa Oil is from 1.5 ml to 3.5 ml per batch of composition. In another embodiment the amount of Palmarosa Oil is 2.5 ml per batch of composition.

In one embodiment the amount of Palo Santo is from 1.0% to 5.0% per batch of composition. In another embodiment the amount of Palo Santo is from 1.5% to 3.5% per batch of composition. In another embodiment the amount of Palo Santo is 2.5% per batch of composition.

In one embodiment the amount of Panthenol is from 1 ml to 20 ml per batch of composition. In another embodiment the amount of Panthenol is from 5 ml to 15 ml per batch of composition. In another embodiment the amount of Panthenol is 10 ml per batch of composition.

In one embodiment the amount of Peppermint is from 1 ml to 10 ml per batch of composition. In another embodiment the amount of Peppermint is from 2.5 ml to 7.5 ml per batch of composition. In another embodiment the amount of Peppermint is 5 ml per batch of composition.

In one embodiment the amount of Proline is from 1 g to 10 g per batch of composition. In another embodiment the amount of Proline is from 2.5 g to 7.5 g per batch of composition. In another embodiment the amount of Proline is 5 g per batch of composition.

In one embodiment the amount of Protocatechuic Acid is from 1 g to 20 g per batch of composition. In another embodiment the amount of Protocatechuic Acid is from 5 g to 15 g per batch of composition. In another embodiment the amount of Protocatechuic Acid is 10 g per batch of composition.

In one embodiment the amount of Rose Hip Seed Oil is from 1 ml to 20 ml per batch of composition. In another embodiment the amount of Rose Hip Seed Oil is from 2.5 ml to 15 ml per batch of composition. In another embodiment the amount of Rose Hip Seed Oil is 5-10 ml per batch of composition.

In one embodiment the amount of Sachi Inchi oil is from 1 ml to 10 ml per batch of composition. In another embodiment the amount of Sachi Inchi oil is from 2.5 ml to 7.5 ml per batch of composition. In another embodiment the amount of Sachi Inchi oil is 5 ml per batch of composition.

In one embodiment the amount of Sea Buckthorn Oil is from 1 ml to 10 ml per batch of composition. In another embodiment the amount of Sea Buckthorn Oil is from 2.5 ml to 7.5 ml per batch of composition. In another embodiment the amount of Sea Buckthorn Oil is 5 ml per batch of composition.

In one embodiment the amount of Sericin is from 10 g to 100 g. In another embodiment the amount of Sericin is from 25 g to 75 g per batch of composition. In another embodiment the amount of Sericin is 50 g per batch of composition.

In one embodiment the amount of Sericin is from 5 g to 50 g. In another embodiment the amount of Sericin is from 10 g to 30 g per batch of composition. In another embodiment the amount of Sodium Polyacrylate is 20 g per batch of composition.

In one embodiment the amount of Tamanu Oil is from 1 ml to 10 ml per batch of composition. In another embodiment the amount of Tamanu Oil is from 2.5 ml to 7.5 ml per batch of composition. In another embodiment the amount of Tamanu Oil is 5 ml per batch of composition.

In one embodiment the amount of Terpineol is from 1 ml to 10 ml per batch of composition. In another embodiment the amount of Terpineol is from 2.5 ml to 7.5 ml per batch of composition. In another embodiment the amount of Terpineol is 5 ml per batch of composition.

In one embodiment the amount of Thyme Red, White is from 1 ml to 10 ml per batch of composition. In another embodiment the amount of Thyme Red, White is from 2.5 ml to 7.5 ml per batch of composition. In another embodiment the amount of Thyme Red, White is 5 ml per batch of composition.

In one embodiment the amount of Vetiver is from 1.0 ml to 5.0 ml per batch of composition. In another embodiment the amount of Vetiver is from 1.5 ml to 3.5 ml per batch of composition. In another embodiment the amount of Vetiver is 2.5 ml per batch of composition.

In one embodiment the amount of Vitamin A is from 1.0 g to 5.0 g per batch of composition. In another embodiment the amount of Vitamin A is from 1.5 g to 3.5 g per batch of composition. In another embodiment the amount of Vitamin A is 2.5 g per batch of composition.

In one embodiment the amount of Vitamin B3 or Niacin is from 1 g to 25 g per batch of composition. In another embodiment the amount of Vitamin B3 or Niacin is from 5 g to 17.5 g per batch of composition. In another embodiment the amount of Vitamin B3 or Niacin is 12.5 g per batch of composition.

In one embodiment the amount of Vitamin C is from 1.0 g to 5.0 g per batch of composition. In another embodiment the amount of Vitamin C is from 1.5 g to 3.5 g per batch of composition. In another embodiment the amount of Vitamin C is 2.5 g per batch of composition.

In one embodiment the amount of Vitamin E is from 1.0 g to 5.0 g per batch of composition. In another embodiment the amount of Vitamin E is from 1.5 g to 3.5 g per batch of composition per batch of composition. In another embodiment the amount of Vitamin E is 2.5 g per batch of composition per batch of composition.

In one embodiment the amount of Winter Savory is from 1 ml to 10 ml per batch of composition. In another embodiment the amount of Winter Savory is from 2.5 ml to 7.5 ml per batch of composition. In another embodiment the amount of Winter Savory is 5 ml per batch of composition.

In one embodiment a method of treatment for Dupuytren's Contracture, comprises administering to a mammal in need thereof a composition comprising a combination of components in Table 1 and 2.

In one embodiment a method of treatment for Skin Damage from Cuts, Abrasions, and Burns, comprises administering to a mammal in need thereof a composition comprising a combination of components in Table 1 and 2.

In one embodiment a method of treatment for Peyronie's Disease, comprises administering to a mammal in need thereof a composition comprising a combination of components in Table 1 and 2.

In one embodiment a method of treatment for Aging Skin, comprises administering to a mammal in need thereof a composition comprising a combination of components in Table 1 and 2.

In one embodiment a method of treatment for Toe Nail Fungus, comprises administering to a mammal in need thereof a composition comprising a combination of components in Table 1 and 2.

In one embodiment a method of treatment for Aphthous Ulcers, comprises administering to a mammal in need thereof a composition comprising a combination of components in Table 1 and 2.

In one embodiment a method of treatment for Scleroderma Induced Subcutaneous Damage, comprises administering to a mammal in need thereof a composition comprising a combination of components in Table 1 and 2.

In one embodiment a method of treatment for Raynaud's Phenomenon, comprises administering to a mammal in need thereof a composition comprising a combination of components in Table 1 and 2.

In one embodiment a method of treatment for Chemotherapy/Radiation Induced Oral Mucosal Ulceration, comprises administering to a mammal in need thereof a composition comprising a combination of components in Table 1 and 2.

Additional embodiments are described in the following paragraphs.

Paragraph 1. A composition comprising α-pinene, ghana shea butter and methylsulfonylmethane.

Paragraph 2. The composition of Paragraph 1 further comprising glycerol, black cumin seed, aloe vera gel, collagen and lecithin.

Paragraph 3. The composition of Paragraph 2 further comprising at least one from the group consisting of functional oil, alcohol, anti-inflammatory, anti-oxidant, anti-microbial, floral, thickener, wax, fragrance, fatty acid, analgesic, amino acid, carbohydrate, vitamin, solvent, gel, moisturizer and acid.

Paragraph 4. The composition of Paragraph 3 wherein the functional oil is selected from the group consisting of Apricot Kernal Oil, Argan Nut Oil, Baobab Oil, Calendula Oil, Camellia Oil, Caprylic, Capric Triglyceride, Caryophyllene, Elemi Oil, Kanuka Oil, Litsea Cubebe Oil, Marula Oil, Palmarosa Oil, Palo Santo, Rose Hip Seed Oil, Sachi Inchi Oil, Sea Buckthorn Oil and Tamanu Oil.

Paragraph 5. The composition of Paragraph 3 wherein the alcohol is selected from the group consisting of Benzyl Alcohol, Cetyl Alcohol and Panthenol.

Paragraph 6. The composition of Paragraph 3 wherein the anti-inflammatory is selected from the group consisting of Acai Berry, Chamomile German Oil, Capaiba Oil, Myrrh, Rose Hip Seed Oil, Oleic Acid and Linoleic Acid.

Paragraph 7. The composition of Paragraph 3 wherein the anti-oxidant is selected from the group consisting of Kaempferol, Caffeic acid and Protocatechuic Acid.

Paragraph 8. The composition of Paragraph 3 wherein the anti-microbial is selected from the group consisting of Anethole Anise Camphor, Cabreuva Oil, Carvacrol, Niaouli Oil, Thyme and Winter Savory.

Paragraph 9. The composition of Paragraph 3 wherein the floral is Rosemary Oil.

Paragraph 10. The composition of Paragraph 3 wherein the thickener is Benzoin.

Paragraph 11. The composition of Paragraph 3 wherein the wax is Bees Wax.

Paragraph 12. The composition of Paragraph 3 wherein the fragrance is selected from the group consisting of Carvone Oil, Geranium Oil, Ho Wood Oil, Jasmine Oil, Lavodin Oil, Limonene Oil, Nerolina Oil, Peppermint, Terpineol and Vetiver.

Paragraph 13. The composition of Paragraph 3 wherein the fatty acid is Ferulic Acid.

Paragraph 14. The composition of Paragraph 3 wherein the analgesic is Benzocaine.

Paragraph 15. The composition of Paragraph 3 wherein the amino acid is selected from the group consisting of Alanine, Arginine, Glutamine, Glycine, Histidine, Hydroxyproline, Isoleucine, Leucine, Lysine and Proline.

Paragraph 16. The composition of Paragraph 3 wherein the carbohydrate is Agave nectar.

Paragraph 17. The composition of Paragraph 3 wherein the vitamin is selected from the group consisting of Vitamin A, Vitamin B3, Niacin, Vitamin C, Vitamin E, Niacinamide.

Paragraph 18. The composition of Paragraph 3 wherein the solvent is distilled water.

Paragraph 19. The composition of Paragraph 3 wherein the gel is Sericin.

Paragraph 20. The composition of Paragraph 3 wherein the moisturizer is selected from the group consisting of Sodium Polyacrylate and 1-Tetradecanol.

Paragraph 21. A method of combining hydrophobic components and hydrophilic components with high dosage of active ingredients, comprising the steps of:
a) placing hydrophobic components that are a liquid or become a liquid at 45° C. in a first vessel and placing hydrophilic components that are a liquid or become a liquid at 45° C. in a second vessel;
b) inserting into each of the first vessel and the second vessel a propeller mixer rotating at 400 rotations per minute (RPM) propeller mixing speed and heating each of the first vessel and the second vessel to 45° C.;
c) after sufficient mixing at 45° C., placing hydrophobic components that are not liquid at 45° C. in the first vessel and placing hydrophilic components that are not liquid at 45° C. in the second vessel, and increasing rotation of each propeller mixer to 800 RPM and increasing the temperature of the first vessel and the second vessel to 60° C.;
d) after sufficient time for mixing and completion of reaction in the first vessel and the second vessel, pouring the contents of the second vessel into the first vessel and combining the contents of both vessels, and maintaining the rotation of the propeller mixer at 800 RPM and temperature at 60° C. for an additional 20 minutes of reaction time;
e) adjusting pH to 7.5 with NaOH;
f) cooling the mixed contents in the first vessel slowly over several hours to ambient temperature by insulating the first vessel and reducing the propeller mixer to 600 RPM;
g) stopping the propeller mixer after the contents in the first vessel reaches ambient temperature;
h) filling mixed content into a container.

Paragraph 22. A method of combining hydrophobic components and hydrophilic components with high dosage of active ingredients, comprising the steps of:
a) placing hydrophobic components that are a liquid or become a liquid at 30° C.-60° C. in a first vessel and placing hydrophilic components that are a liquid or become a liquid at 30° C.-60° C. in a second vessel;
b) inserting into each of the first vessel and the second vessel a propeller mixer rotating at 200-600 rotations per minute (RPM) propeller mixing speed and heating each of the first vessel and the second vessel to 30° C.-60° C.;
c) after sufficient mixing at 30° C.-60° C., placing hydrophobic components that are not liquid at 30° C.-60° C. in the first vessel and placing hydrophilic components that are not liquid at 30° C.-60° C. in the second vessel, and increasing rotation of each propeller mixer to 600-1,000 RPM and increasing the temperature of the first vessel and the second vessel to greater than or equal to 60° C.;
d) after sufficient time for mixing and completion of reaction in the first vessel and the second vessel, pouring the contents of the second vessel into the first vessel and combining the contents of both vessels, and maintaining the rotation of the propeller mixer at 600-1,000 RPM and temperature at greater than or equal to 60° C. for an additional 10-60 minutes of reaction time;
e) adjusting pH to 7.5 with NaOH;
f) cooling the mixed contents in the first vessel slowly over several hours to ambient temperature by insulating the first vessel and reducing the propeller mixer speed to 400-800 RPM;
g) stopping the propeller mixer after the contents in the first vessel reaches ambient temperature;
h) filling mixed content into a container.

Paragraph 23. The method of combining hydrophobic components and hydrophilic components with high dosage of active ingredients in Paragraph 22, wherein the temperature in step a) is 40° C.-50° C.

Paragraph 24. The method of combining hydrophobic components and hydrophilic components with high dosage of active ingredients in Paragraph 23, wherein the temperature in step a) is 45° C.

Paragraph 25. The method of combining hydrophobic components and hydrophilic components with high dosage of active ingredients in Paragraph 22, wherein the propeller mixing speed is step b) is 300-500 RPM.

Paragraph 26. The method of combining hydrophobic components and hydrophilic components with high dosage of active ingredients in Paragraph 25, wherein the propeller mixing speed is step b) is 400 RPM.

Paragraph 27. The method of combining hydrophobic components and hydrophilic components with high dosage of active ingredients in Paragraph 22, wherein the temperature in step b) is 40° C.-50° C.

Paragraph 28. The method of combining hydrophobic components and hydrophilic components with high dosage of active ingredients in Paragraph 27, wherein the temperature in step b) is 45° C.

Paragraph 29. The method of combining hydrophobic components and hydrophilic components with high dosage of active ingredients in Paragraph 22, wherein the temperature in step c) is 40° C.-50° C.

Paragraph 30. The method of combining hydrophobic components and hydrophilic components with high dosage of active ingredients in Paragraph 29, wherein the temperature in step c) is 45° C.

Paragraph 31. The method of combining hydrophobic components and hydrophilic components with high dosage of active ingredients in Paragraph 22, wherein the propeller mixing speed is step c) is 700-900 RPM.

Paragraph 32. The method of combining hydrophobic components and hydrophilic components with high dosage of active ingredients in Paragraph 31, wherein the propeller mixing speed is step c) is 800 RPM.

Paragraph 33. The method of combining hydrophobic components and hydrophilic components with high dosage of active ingredients in Paragraph 22, wherein the temperature in step c) is increased to 60° C.-80° C.

Paragraph 34. The method of combining hydrophobic components and hydrophilic components with high dosage of active ingredients in Paragraph 33, wherein the temperature in step c) is increased to greater than 80° C.

Paragraph 35. The method of combining hydrophobic components and hydrophilic components with high dosage of active ingredients in Paragraph 22, wherein the propeller mixing speed is step d) is 700-900 RPM.

Paragraph 36. The method of combining hydrophobic components and hydrophilic components with high dosage of active ingredients in Paragraph 35, wherein the propeller mixing speed is step d) is 800 RPM.

Paragraph 37. The method of combining hydrophobic components and hydrophilic components with high dosage of active ingredients in Paragraph 22, wherein the temperature in step d) is increased to 60° C.-80° C.

Paragraph 38. The method of combining hydrophobic components and hydrophilic components with high dosage of active ingredients in Paragraph 37, wherein the temperature in step d) is increased to greater than 80° C.

Paragraph 39. The method of combining hydrophobic components and hydrophilic components with high dosage of active ingredients in Paragraph 22, wherein the sufficient time is 1 to 8 hours.

Paragraph 40. The method of combining hydrophobic components and hydrophilic components with high dosage of active ingredients in Paragraph 39, wherein the sufficient time is more than 8 hours.

Paragraph 41. The method of combining hydrophobic components and hydrophilic components with high dosage of active ingredients in Paragraph 22, wherein the additional reaction time is 10-40 minutes.

Paragraph 42. The method of combining hydrophobic components and hydrophilic components with high dosage of active ingredients in Paragraph 41, wherein the additional reaction time is 20 minutes.

Paragraph 43. The method of combining hydrophobic components and hydrophilic components with high dosage of active ingredients in Paragraph 22, wherein the several hours in step f) is 2 to 10 hours.

Paragraph 44. The method of combining hydrophobic components and hydrophilic components with high dosage of active ingredients in Paragraph 43, wherein the several hours in step f) is more than 10 hours.

Paragraph 45. The method of combining hydrophobic components and hydrophilic components with high dosage of active ingredients in Paragraph 22, wherein the propeller mixer speed in step f) is 500-700 RPM.

Paragraph 46. The method of combining hydrophobic components and hydrophilic components with high dosage of active ingredients in Paragraph 46, wherein the propeller mixer speed in step f) is 600 RPM.

Paragraph 47. The method of combining hydrophobic components and hydrophilic components with high dosage of active ingredients in Paragraph 22, wherein the ambient temperature in step g) is 25° C.-37° C.

Paragraph 48. The method of combining hydrophobic components and hydrophilic components with high dosage of active ingredients in Paragraph 47, wherein the ambient temperature in step g) is less than 25° C.

The disclosed compositions for treatments of topical diseases and syndromes, many with similarities in terms of their anti-inflammatory and anti-oxidative activities, but also multiple differences in their observed abilities that can be combined to challenge the current, underlying pathophysiology. The following disorders, described below, can be treated with the disclosed compositions with the proper combination and alteration of ingredients:

1. Dupuytren's Contracture

2. Skin Damage from Cuts, Abrasions, and Burns

3. Peyronie's Disease

4. Aging Skin Changes on arms and hands

5. Toe Nail Fungus

6. Aphthous Ulcers

7. Scleroderma Induced Subcutaneous Damage

8. Raynaud's (or Renaud's) Phenomenon

9. Chemotherapy/Radiation Induced Oral Mucosal Ulceration

### BRIEF DESCRIPTION OF THE SEVERAL VIEWS OF THE DRAWING

FIG. 1 shows a hand with a chord of a patient with Dupytren's Disease and Contracture [permission granted—Frank C. Müller, Baden-Baden].

FIG. 2A shows an abrasion of the palm.

FIG. 2B shows a cut on the leg [permission granted—ClockFace].

FIG. 2C shows a burn to the top of a hand.

FIG. 3 illustrates the curvature of the penis due to Peyronie's Disease.

FIG. 4A shows damage to the skin of the forearm of an elderly person.

FIG. 4B is a graph of age range of the United States, Year 2016.

FIG. 5 shows toes with severe fungus infection of toe nails [permission granted—James Heilman, MD].

FIG. 6 shows an aphthous ulcer in the mouth of a patient [permission granted—Ryanfransen at English Wikipedia].

FIG. 7 shows several photographs of people with cutaneous scleraderma

FIG. 8 illustrates the effects of Raynaud's phenomenon on the hand.

FIG. 9 shows the ulcers Induced in Oral Mucosa by Chemotherapy/Radiation Therapy.

### DETAILED DESCRIPTION OF THE INVENTION

### Components of the Compositions

Table 3 shows the function, and scientific name or chemical formula for each hydrophobic component that can be used in a composition.

TABLE 3

| Hydrophobic Components | Function | Scientific name/Formula |
|---|---|---|
| α-Pinene (APN) | anti-inflammatory, anti- | Pinophyta |
| | microbe | C₁₀H₁₆ |
| Acai Berry | anti-inflammatory | Euterpe oleracea |
| Anethole Anise Camphor | anti-microbial | C₁₀H₁₂O |
| Apricot Kernel Oil (AKO) | functional oil | Prunus armeniaca |
| Argan Nut Oil (AGN) | skin disorders | Argania spinosa |
| Baobab Oil (BAB) | moisturizer | Adansonia digitata |
| Bees Wax (BW) | phase change emulsion | Apis mellifera |
| Benzoin (BZN) | thickener | Styrax benzoin |
| Benzyl Alcohol (BA) | solvent, low toxicity | C₆H₅CH₂OH |
| Black Cumin Seed | oil | Nigella sativa |
| Cabreuva Oil | anti-microbial | Myrocarpus frondosus |
| Caffeic acid | anti-oxidant | C₉H₈O₄ |
| Calendula Oil (CDA) | anti-inflammatory | Calendula officinalis |
| Camellia Oil (CMA) | anti-oxidant | Camellia japonica |
| Capaiba Oil (CPO) | anti-inflammatory | Copaifera officinalis |
| Caprylic, Capric Triglyceride | soft skin | Cocos nucifera |
| (CCT) | | |
| Carvacrol (CVC) | anti-microbial | C₁₀H₁₄O |
| Carvone Oil (CRV) | fragrance | C₁₀H₁₄O |
| Caryophyllene (CAP) | anti-oxidant | Syzygium aromaticum |
| Cedarwood essential Oil | flavor | Pine or cypress |
| Cetyl Alcohol (CTA) | alcohol | C₁₆H₃₄O |
| Chamomile German Oil (CGB) | fragrance | Chamaemelum nobile |
| Elemi Oil (EMO) | functional oil | Canarium luzonicum |
| Geranium Oil (GER) | essential oil | Pelargoniun graveolens |
| Ghana Shea Butter (GSB) | salve, moisturize | Vitellaria paradoxa |
| Glycerol (GYO) | moisturizer, solvent | C₃H₈O₃ |
| Ho Wood Oil (HWO) | fragrance | Cinnamomum camphora |
| Jasmine Oil (JAS) | essential oil | Jasminum officinale |
| Kaempferol | anti-oxidant | C₁₅H₁₀O₆ |
| Kanuka Oil (KNO) | functional oil | Kunzea ericoides |
| Lavandin Oil (LVO) | fragrance | Lavandin intermedia |
| Limonene Oil (LMN) | fragrance | C₁₀H₁₆ |
| Litsea Cubeba Oil (LCO) | functional oil | Litsea cubeba |
| Marula Oil (MRL) | moisture, anti-oxidant | Sclerocarya birrea |
| Myrrh | anti-inflammatory | Genus Commiphora |
| Nerolina Oil (NRL) | fragrance | Melaleuca quinquenervia |
| Niaouli Oil | anti-microbial | Melaleuca quinquenervia |
| Palmarosa Oil (PLO) | traditional, anti-microbial | Cymbopogon martinii |
| Palo Santo | functional oil | Bursera graveolens |
| Panthenol (PNT) | alcohol | C₉H₁₉NO₄ |
| Peppermint | fragrance | Mentha × piperita |
| Rose Hip Oil (RHO) | healing, moisturizing | Rosa moschata & Rosa |
| | | rubiginosa |
| Rosemary Oil (RMO) | floral | Rosmarinus officinalis, |
| Sacha Inchi Oil (SIO) | supportive skin care | Plukenetia volubillis |
| Sea Buckthorn Oil (SBO) | repair, anti-oxidant | Hippophae rhamnoides |
| Tamanu Oil (TMU) | skin care | Calophyllum inophyllum |
| Terpineol | fragrance | C₁₀H₁₈O |
| Thyme Red, White | anti-microbial | Genus Thymus |
| Vetiver | fragrance | Chrysopogon zizanioides |
| Vitamin E (VTE) | cell reactions, anti-oxidant | Tocotrienol, tocopherol |
| Winter Savory | anti-microbial | Satureja montana |

Table 4 shows the function, and scientific name or chemical formula for each hydrophilic component that can be used in a composition.

TABLE 4

| Hydrophilic Components | Function | Scientific name |
|---|---|---|
| 1-Tetradecanol | moisturizer | C₁₄H₃₀O |
| Agave (AGV) | cell nutrients, fructose, sucrose | Agave tequilana weber azul |
| Alanine (ALA) | amino acid for collagen | C₃H₇NO₂ |
| Aloe Vera Gel (AVG) | skin healing, anti- | Aloe barbadensis mil |
| | inflammatory | |
| Arginine (ARG) | amino acid | C₆H₁₄N₄O₂ |
| Artic Fish Collagen (AFC) | critical soluble protein | Arctogadus glacialis |
| Benzocaine (BZC) | local analgesic | C₉H₁₁NO₂ |
| Distilled water | solvent | H₂O |
| Ferulic Acid (FRA) | anti-oxidant | C₁₀H₁₀O₄ |
| Glutamine (GLU) | amino acid | C₅H₁₀N₂O₃ |
| Glycine (GLY) | amino acid for collagen | C₂H₅NO₂ |
| Histidine (HST) | amino acid | C₆H₉N₃O₂ |
| Hydroxyproline (HDP) | amino acid | C₅H₉NO₃ |
| Isoleucine (ISL) | amino acid | C₆H₁₃NO₂ |
| Lecithin (LCT) | skin soft and smooth | Glycine max |
| Leucine (LUC) | amino acid | C₆H₁₃NO₂ |
| Linoleic Acid (LOA) | anti-inflammatory | C₁₈H₃₂O₂ |
| Lysine (LYS) | amino acid | C₆H₁₄N₂O₂ |
| Methyl sulfonyl methane | transport | C₂H₆SO₂ |
| (MSM) | | |
| Niacin (NCN) | vitamin b3 skin health, | C₆H₅NO₂ |
| | vasodilate | |
| Niacinamide (NCM) | vitamin b3, no flush | C₆H₆N₂O |
| Oleic Acid (OLA) | anti-inflammatory | C₁₈H₃₄O₂ |
| Panthenol | alcohol analogue of | C₉H₁₉NO₄ |
| | Vitamin B5 | |
| Proline (PRL) | amino acid for collagen | C₅H₉NO₂ |
| Protocatechuic Acid | anti-oxidant | C₇H₆O₄ |
| (PCT) | | |
| Sericin | gel | C₃₀H₄₀N₁₀O₁₆ |
| Sodium Polyacrylate | moisturizer | (C₃H₃NaO₂)ₙ |
| ethanol | solvent | C₂H₆O |
| isopropyl alcohol | solvent | C₃H₈O |
| Vitamin A (VTA) | vitamin | retinol, retinal, |
| | | retinoic acid, provitamin A |
| | | carotenoids, beta-carotene |
| Vitamin B3 (VTB3) or | vitamin b3 skin health, | nicotinamide, niacin, |
| Niacin | vasodilate | nicotinamide riboside |
| Vitamin C (VTC) | tissue repair, anti-oxidant | C₆H₈O₆ |

### Hydrophobic Components

Acai Berry—A small, round, black-purple fruit similar in appearance to a grape. The seed makes up about 60-80% of the fruit. The palm bears fruit year round.

α-Pinene—Formula—C₁₀H₁₆—α-Pinene is a terpene that is not toxic, which is one of two isomers of this compound with the other being β-Pinene that has toxic characteristics. They originate predominantly from conifer pine trees (Pinophyta) and are both natural compositions. α-Pinene has important anti-inflammatory properties working through the PGE1 mechanism. In addition, it has important anti-microbial activities. It also been used in medicinal compositions as a bronchial dilator, anti-septic agent, and anti-cancer agent. Its primary use in the composition is its anti-inflammatory and anti-microbial effects.

Anethole Anise Camphor—Formula—C₁₀H₁₂O—An organic compound that is widely used as a flavoring. A derivative of phenylpropene, an aromatic compound that occurs widely in nature in essential oils. It is distinctly sweet and pleasant to the taste. It contributes a large component of the odor and flavor of anise and fennel. It is a colorless, fragrant, mildly volatile liquid. Anethole has potent antimicrobial properties. Reported antibacterial properties include both bacteriostatic and bactericidal action. Antifungal activity includes increasing the effectiveness of some other phytochemicals.

Apricot Kernal Oil (AKO)—Pressed from the kernels of the Prunus armeniaca. Apricot kernels have an oil content of 40-50%. It is also used to extract an essential oil, which contains amygdalin. The oil is chiefly composed of oleic acid and linoleic acid.

Argan Nut Oil—Argania spinosa is an important tree grown in Morocco. It is a dietary oil and used in cosmetics. The oil comes from the tree's nuts. Its intended use for the compositions is to support the oils with higher levels of healing.

Baobab Oil—Adansonia digitalia is the most common of the nine species of Baobab trees with the oil extracted from seeds. It is commonly used on the skin as a moisturizer, a stimulant for skin cell replication, a skin anti-oxidant, a wound healing agent, and an anti-inflammatory agent due to its omega fatty acids. Is use in the compositions is primarily for its anti-oxidant, anti-inflammatory, and wound healing properties.

Bees Wax—Wax from honey bees (Apis mellifera) is utilized in some of the compositions as a temperature sensitive agent that melts at 58° C. and solidifies at lower temperatures depending upon concentration that keeps the manufacturing process in the liquid phase for alterations and modifications of the natural components at a higher temperature. Upon cooling, it acts with other components to provide solidity within the final composition.

Benzoin (BZN)—Styrax benzoin—A balsamic resin obtained from the bark of several species of trees in the genus Styrax. It is used in perfumes, incense, flavoring, and medicine. Benzoin is sometimes called gum benzoin or gum benjamin. Benzoin is also called storax. Benzoin has a sweet vanilla-like aroma and fixative properties.

Benzyl Alcohol—Formula C₆H₅CH₂OH—Benzyl Alcohol is often used as a solvent due to low toxicity, polarity, and low vapor pressure with moderate solubility in water and miscibility in other alcohols. It is commonly found in plants and fruits and along with a variety of essential oils, such as, jasmine, hyacinth, and ylang-ylang. It is also used as a bacteriostatic preservative in intravenous medications, cosmetics, and skin care compositions. It has a low toxicity value with LD₅₀ of 1.2 gm/kg in rats. Its use in the compositions is as a solvent and its anti-microbial properties.

Black Cumin Seed Oil (BCS)—Nigella sativa—It is an annual flowering plant in the family Ranunculaceae.

Cabreuva Oil—Myrocarpus frondosus—Its aroma is deep and mellow, with a pleasing woodsy, smoky base note that blends very well with most oils. In natural perfumery, clove bud essential oil is used in making floral notes such as rose and lily of the valley, amber bases, precious wood notes, and in

Caffeic acid—Formula—C₉H₈O₄— An organic compound classified as a hydroxycinnamic acid. It consists of both phenolic and acrylic functional groups. It is found in all plants because it is a key intermediate in the biosynthesis of lignin. Caffeic acid has a variety of potential pharmacological effects and the inhibitory effect of caffeic acid on cancer cell proliferation by an oxidative mechanism. Caffeic acid is an antioxidant, immunomodulatory and anti-inflammatory activity.

Calendula Oil—Calendula officinalis is a type of marigold that is used for herbal, cosmetic, and medicinal compositions. The yellow petals are edible and have been used medicinally for centuries. It is currently utilized for its pharmacologic effects for anti-inflammatory, anti-tumor, and wound healing attributes. Topical applications are also used on the skin for radiation damage, the prevention of dermatitis and pain, as well as, an agent to decrease healing times on skin injuries. It was a popular treatment in the Civil War and World War I to reduce wound bleeding and infection. Its use in the compositions is for its anti-inflammatory and wound healing properties.

Camellia Oil—Camellia japonica has been used for centuries by the Japanese as skin care and moisturizing compositions, as well as, for wound healing and scar reduction as it readily absorbs into the skin. It has also been used medically as a vitamin carrier for injections. The oil is cold pressed from seeds. Camellia is part of the flowering plants from the Theaceae family with leaves from Camellia sinensis the most popular for tea products. Its primary use in the compositions is it anti-inflammatory and wound healing properties.

Capaiba Oil (CPO)—Copaifera officinalis—It reduces pain and inflammation, protects against infections, heals the skin, prevents fungal growth, and boosts the respiratory health. It improves the skin, speeds up healing, and tightens the skin. Copaiba essential oil is a distilled oil from an oleoresin. This resin is produced as a sap from a tree in the Copaifera genus. The oil is a pale yellow in color with a slightly bitter taste and an aroma. Copaiba is considered to be one of the most anti-inflammatory substances on earth. In traditional medicine, this essential oil has been used extensively by indigenous people. The active components in copaiba essential oil consist mainly of terpenes. It has topical applications and the copaiba essential oil has astringent properties. Copaiba's active components tighten the skin, which can help the skin look younger, reduce the appearance of wrinkles, and even strengthen the skin to prevent lesions or wounds as the body ages. Additionally, a topical application of copaiba essential oil helps reduce the appearance of acne on the face. Copaiba essential oil also heals the skin and infuses the body with powerful nutrients and organic compounds that can eliminate the appearance of blisters, marks, and pimples. Copaiba essential oil is often topically applied to scars in order to speed their healing and reduce their visibility. Copaiba essential oil is a analgesic and is a strong antibacterial agent. It can act as a shield for your skin, protecting any wounds from developing an infection. The unique terpene structures found in copaiba essential oil are very effective as antifungal agents.

Capryllic, Capric Triglyceride—Formulae—Capryllic Acid C₈H₁₆O₂,—Capric Acid CH₃(CH₂)₈COOH—Cocos nucifera is the cocoa nut palm from which the composition is purified as <10% constituents of the kernal. Individual uses of these triglycerides have little direct effect on the intended use of this topical composition. However, this mixed, medium chain triglyceride composition is considered an inert energy source readily available for use by the skin cells and dermis cells that are being challenged to function properly in their limited condition brought on by their specific disease state.

Carvacrol (CVC)—Formula—C₆H₃(CH₃)(OH)C₃H₇— It is a monoterpenoid phenol. It has a characteristic pungent, warm odor of oregano. Carvacrol inhibits the growth of several bacteria strains, e.g. Escherichia coli. It disrupts cell membranes of bacteria and inhibits their proliferation.

Carvone Oil (CRV)—Formula—C₁₀H₁₄O—It is a terpenoid and found naturally in many essential oils, but is most abundant in the oils from seeds of caraway (Carum carvi), spearmint (Mentha spicata), and dill. Carvone is available in both enantiomerically pure forms.

Caryophyllene Oil—This oil is most commonly from the stem and flowers of Syzygium aromaticum to produce clove oil. There are 14 plant sources of Caryophyllene oil. However, only 9 of these plants have their Caryophyllene levels above 10% of these oils from clove, cannabis, hops, basil, oregano, West African pepper, cinnamon, malabathrum, and ylang-ylang. Two of these, the West African pepper and the India cinnamon or malabathrum, contain up to 25% of their oils as Caryophyllene oil. Its use in the compositions is primarily for its anti-inflammatory properties.

Cedarwood essential Oil—An essential oil derived from various types of conifers, most in the pine or cypress botanical families. It has uses in medicine and perfumery, and while the characteristics of oils derived from various species may themselves vary, all have some degree of bactericidal effects. Although termed cedar or cedarwood oils, the most important oils are produced from distilling wood of a number of different junipers and cypresses (of the family Cupressaceae), rather than true cedars (of the family Pinaceae). Cedarwood oils each have characteristic woody odors which may change somewhat in the course of drying out. The crude oils are often yellowish or even darker in color. They find use in fragrance applications. The oil has antifungal and antibacterial properties.

Cetyl Alcohol (CTA)—Formula—CH₃ (CH₂)₁₅OH—It is a fatty alcohol and also known as hexadecan-1-ol and palmityl alcohol. At room temperature, cetyl alcohol takes the form of a waxy white solid or flakes. Cetyl alcohol is used in the cosmetic industry as an opacifier in shampoos, or as an emollient, emulsifier or thickening agent in the manufacture of skin creams and lotions.

Chamomile German Oil—Chamaemelum nobile is used for treating skin conditions such as eczema, chicken pox lesions, and psoriasis. It has a wide medical usage for inflammation, ulcers, and hemorrhoids. Chamomile tea has long been popular and is made from the dried flowers and may be a relaxant. There also may be anti-anxiety effects from its use. Its primary use in the compositions is to aid healing and reduce inflammation.

Elemi Oil (EMO)—It is steam distilled from the resin of the Asian tree Canarium luzonicum or Canarium vulgare. Elemi Oil is rich in monoterpenes. It can be especially helpful in wound care and for supporting respiratory health. It has a clear color with a tinge of yellow. It is effective against infectious skin conditions, wounds, cuts, fatigue.

Geranium Essential Oil—Pelargoniun graveolens encompasses a relatively large group of plants from which this complex, essential oil comprises ˜50 different oil components. The number of species has been greatly increased due to significant hybrid culturing seeking to focus on different flower and scent expressions. It currently has limited medicinal use and is being used as a general oil solution and fragrance for these applications.

Ghana Shea Butter—Vitellaria paradoxa is the general name for the Shea trees of the Sapotacea family and is the only species in this genus, located predominantly in Africa. This species is a major food source of dietary fat. Shea butter is composed of 5 fatty acids: palmitic, stearic, oleic, linoleic and arachidic of which 85-90% is stearic and oleic acids. These fatty acid contents change in percentages from region to region in Africa with the shea butter changing from liquid to near solid depending upon the fatty acid concentrations. There are also 10 phenolic compounds in shea butter that also vary from region to region. Therefore, shea butter is obtained from one region, Ghana. Shea butter is one of the largest components of the compositions and is used not only for composition consistency but also as a major anti-oxidant.

Glycerol—Formula—C₃H₈O₃— Glycerol is a colorless, odorous liquid with three —OH groups assuring solubility in water that is also hydroscopic. It is the backbone of all triglycerides. It is derived from soy beans and under large scale manufacturing. It is widely used for extractions of plant compositions and as a solvent. Its primary use in the compositions is as a solvent.

Ho Wood Oil (HWO)—Cinnamomum camphora var linaloo—It is steam distilled from the bark and wood. Ho Wood is one of the most potent sources of naturally occurring linalol found in any steam distilled essential oil. Aromatically, Ho Wood Essential Oil is a beautifully fragrant wood oil that possesses some similarity to that of Rosewood Oil. The color is clear. A sweet, fresh and woody, with subtle floral notes. Its significant content of linalol gives it analgesic, anti-inflammatory and antibacterial properties.

Jasmine Essential Oil—Jasminum officinale is a species of the olive family in Eastern Europe and Asia that has been adopted as a garden varietal with appealing flowers. It has a history of use in aromatherapy and herbal medicine predominantly as a skin anti-septic and anti-inflammatory agent. Its inclusion in the compositions is for these uses, as well as, its essential oil fragrance.

Kaempferol—Formula—3,4′,5,7-tetrahydroxyflavone (C₁₅H₁₀O₆)—It is a natural flavonol. Kaempferol is a yellow crystalline solid. It is slightly soluble in water and highly soluble in hot ethanol, ethers, and DMSO. Kaempferol acts as an antioxidant by reducing oxidative stress. Kaempferol is common in Pteridophyta, Pinophyta and Angiospermae. Studies have shown kaempferol has pharmacological activities, including antioxidant, anti-inflammatory, antimicrobial, and analgesic activities. It also has been shown to work synergistically with antibiotics. Kaempferol has been shown to inhibit or decrease the activity of enzymes in viral infection, such as, reverse transcriptase, viral proteases and neuraminidase. Kaempferol has been shown to have an array of antioxidant effects. Kaempferol can prevent the oxidation of low-density lipid proteins.

Kanuka Oil (KNO)—Kunzea ericoides/Leptospermum ericoides—Sometimes known as White Tea Tree Essential Oil. Kanuka is much higher in monoterpenes than is Australian Tea Tree. It contains about 5% cineole. It is a pale yellow color.

Lavandin Oil (LVO)—The two most popular varieties are Lavandula angustifolia (lavender) and Lavandula Intermedia (lavandin). Lavandin is also commonly referred to as French lavender because it was developed for the French perfume industry. It is considered to be an antiseptic. Lavandin is a hybrid plant of true lavender and spike lavender. The oil is a pale yellow color to an almost neutral hue. The essential oil may assist in removing scars and stretch marks. It enhances blood circulation, promotes cell regeneration, and heals wounds and cuts. Lavandin oil has antiseptic properties that helps prevent infections from lacerations. It also speeds up the healing of cuts. It relieves pain and inflammation. The essential oil may alleviate pain in the muscles and joints. Lavandin oil is extracted by steam distilling the plant stalks and flowers. Lavandin may also be steam distilled with the help of a volatile solvent like benzene.

Limonene Oil (LMN)—Formula—C₁₀H₁₆—It is a colorless liquid aliphatic hydrocarbon classified as a cyclic monoterpene, and is the major component in the oil of citrus fruit peels. The D-isomer; occurring more commonly in nature as the fragrance of oranges, is a flavoring agent in food manufacturing. Limonene takes its name from the peel of the lemon. Limonene is a chiral molecule, and biological sources produce one enantiomer. Limonene is a relatively stable monoterpene and can be distilled without decomposition. It is a fragrance ingredient for cosmetics compositions.

Litsea Cubeba Oil (LCO)—Litsea cubeba is an evergreen tree or shrub in the Lauraceae family. It produces a fruit which is processed for its lemony essential oil. The oil is used as a fragrance and flavoring. It is used as a raw material by the chemical industry for the synthesis of vitamin A and violet-like fragrances.

Marula Oil—Sclerocarya birrea produces a nut from which this oil is recovered. Marula Oil contains a large proportion of mono-unsaturated fatty acids, as well as, significant anti-oxidants. Its primary anti-oxidant effects are due to tocopherols, sterols, and flavonoids. The primary use in the compositions is its wide types of fatty acids, as well as, its anti-oxidants.

Myrrh—It is a natural gum or resin extracted from a number of small, thorny tree species of the genus Commiphora. Myrrh resin is used as a perfume, incense, medicine, antiseptic and salves for abrasions and other minor skin ailments.

Nerolina Oil (NRL)—Melaleuca quinquenervia—It is a sweetly-scented oil distilled from the leaves and small branches of the paperbark tea tree or the broad-leaved paperbark tree. Nerolina oil is a component of perfume and has topical antibacterial properties. Nerolina oil is a rich emollient and easily penetrates the skin's surface. It can be added to coconut oil, cocoa butter, shea butter or other natural moisturizers to accentuate their effects to soften skin. Nerolina oil aids in the composition ion of vitamin E and the skin proteins collagen and elastin, which make up the skin's structural layer and accelerates cell turnover.

Niaouli Oil—It is also extracted from Melaleuca quinquenervia. Niaouli Oil has camphorous, earthy and a harsh smell. It has therapeutic benefits helpful for skin infections, acne, rashes, pimples, wounds and cuts.

Palmarosa Essential Oil—Cymbopogon martinii—It is a species of lemon grass with a popular fragrance. It contains geroniol and is used in medicinal applications. It is an effective anti-fungal agent, and used predominantly in food storage. Its primary use in the compositions is its fragrance but also as an anti-fungal and potential non-specific medicinal actions.

Palo Santo—Also called Lignum vitae, guayacan or guaiacum. The wood is obtained chiefly from Guaiacum officinale and Guaiacum sanctum, both small, slow growing trees. “Lignum vitae” is Latin for “wood of life,” and derives its name from its medicinal uses. Lignum vitae resin is used to treat a variety of medical conditions from coughs to arthritis.

Panthenol (PNT)—Formula—HO—CH₂—C(CH₃)₂—CH(OH)—CONH—CH₂CH₂CH₂—OH—The alcohol analog of pantothenic acid (vitamin B₅) and a provitamin of B₅. It is commonly utilized in pharmaceuticals, cosmetics, and skin care products as a moisturizer and humectant. It readily penetrates the skin and mucous membranes where it is converted to pantothenic acid that readily binds water and enables reactivity with other composition components and with the tissues. It is odorless, colorless and a viscous liquid at room temperature. Panthenol is to improve wound healing in pharmaceutical and cosmetic compositions. In ointments it is used for the treatment of sunburns, mild burns, and minor skin injuries. It improves hydration, reduces itching and inflammation of the skin, improves skin elasticity, and accelerates epidermal wounds' rate of healing. Pantothenic acid is extremely hygroscopic. It is used in the biosynthesis of coenzyme A. Panthenol is an odorless, slightly bitter, highly viscous, transparent and colorless liquid at room temperature. It is easily soluble in water. Panthenol comes in two enantiomers, D and L. Only D-panthenol (dexpanthenol) is biologically active, however both forms have moisturizing properties.

Peppermint—Mentha×piperita—An herbal extract using alcohol of essential oils of peppermint leaves. Medicinal uses of peppermint extract may relieve itching when applied topically. Moreover, peppermint extract is believed to have antiviral and medicinal properties.

Rose Hip Oil—Rosa moschata, R. rubiginosa and R. canina—Wild rose species that produce seeds are extracted for Rose Hip Oil. It contains Provitamin A with high levels of tretinoin that is an all-trans retinoic acid that can be converted to Vitamin A. Rose Hip Oil contains the essential fatty acids of linoleic acid (omega 6) and linoleic acid (omega 3). Rose Hip Oil is frequently used in a variety of skin conditions including eczema, dermatitis, and sunburn, as well as, photoaging and healing scars. Its inclusion in these formulations is primarily for its anti-inflammatory effects.

Rosemary Oil (RMO)—Rosmarinus officinalis—It is a woody, perennial herb with fragrant flowers and is a member of the mint family Lamiaceae. Rosemary leaves are used as a flavoring in foods. Rosemary oil is used for fragrant bodily perfumes and an aroma into a room.

Sacha Inchi Oil—Plukenetia volubillis—It is a small tree that produces star shaped fruits with enclosed nuts. The nuts contain high protein (27%) and oil (35-60%). The oil is rich in essential fatty acids. There is not much evidence for its use in skin care compositions but its components suggest it should be helpful in skin care compositions. Its primary use in the compositions is supportive of the other oils with already demonstrated effectiveness.

Sea Buckthorn Oil—Hippophae rhamnoides—It accumulates lipids in the fleshy, pulp part of the fruit, as well as, in their seeds. The pulp oil is preferred for these applications since it has high concentrations of the mono-unsaturated fatty acid, palmitoleic acid, and the saturated fatty acid, palmitic acid, which are at unusual levels compared to the plant kingdom. Caretenoids provide the color in the oil and provide beta-carotene, zeazanthin, and lycopene that are associated with Vitamin A activity. The tocopherols and tocotrienols components are major reasons for the anti-oxidant activity of this oil. Its primary purpose for the compositions is its anti-oxidant activities.

Tamanu Oil—Calophyllum inophyllum—It is a large evergreen that produces nuts that contain 70-75% oil. This oil contains Linoleic acid (38%), Oleic acid (34%), Stearic acid (13%), and Palmitic acid (12%). Additional oil components include calophyllolide, freidlin, inophyllums B & P, terpenic essences, benzoic acid, oxibenzoic acid, phospho-amino lipids, glycerides, saturated fatty acids, and 4-phenylcoumarins. This oil has been used for skin diseases and for treating ulcers and infected wounds. Its inclusion in the compositions is for its anti-inflammatory and wound healing characteristics.

Terpineol—Formula—C₁₀H₁₈O—It is a monoterpene alcohol that has been isolated from a variety of sources such as cajuput oil, pine oil, and petitgrain oil. There are four isomers, alpha-, beta-, gamma-terpineol, and terpinen-4-ol. Beta- and gamma-terpineol differ only by the location of the double bond. Terpineol is usually a mixture of these isomers with alpha-terpineol as the major constituent. Terpineol has a pleasant odor similar to lilac.

Thyme Red, White—An aromatic perennial evergreen herb with culinary, medicinal, and ornamental uses. The most common variety is Thymus vulgaris. Thyme is of the genus Thymus of the mint family (Lamiaceae). Thymol, an antiseptic, is an active ingredient in various commercially produced mouthwashes. Oil of thyme was used to medicate bandages before the advent of modern antibiotics.

Vetiver—Chrysopogon zizanioides—It is a perennial bunchgrass of the Poaceae family. Vetiver has been used to produce perfumes, creams and soaps. It is used for its antiseptic properties to treat acne and sores. Vetiver is mainly cultivated for the fragrant essential oil distilled from its roots. Vetiver is a more common ingredient in fragrances for men.

Vitamin E—Formula (α-tocopherol)—C₂₉H₅₀O₂—Vitamin E has several different configurations, but the most prominent is α-tocopherol. Vitamin E is a lipid soluble vitamin from wheat germ oil, sunflower oil, and safflower oil. It stops the propagation of oxygen radicals through cell membranes and protects cells from oxygen radical damage. It is used in a number of medical supplements and topical medication, but it may not be effective in wound healing or reduction of scar tissue. Its use in the compositions is to provide assistance in proper healing of wounds and its anti-oxidant properties.

Winter Savory—Satureja montana—It is a perennial, semi-evergreen herb in the family Lamiaceae. Winter savory has antiseptic and aromatic benefits. It has been used in the treatment of bee stings or insect bites. Therapeutic-grade oil inhibits growth of yeast.

### Hydrophilic Components

1-Tetradecanol—Formula—C₁₄H₃₀O—Myristyl alcohol (Myristica fragrans—nutmeg plant) is a straight-chain saturated fatty alcohol. 1-Tetradecanol is prepared by the hydrogenation of myristic acid (or its esters). Myristic acid is found in nutmeg. 1-Tetradecanol is used as an ingredient in cosmetics, such as, cold creams for its emollient properties.

Agave Nectar—Agave tequilana weber azul—Besides producing tequila, it produces agave nectar that is high in fructose (47%) and glucose (16%) concentrations. It is included in the compositions as a readily available fuel source for the cells being treated.

Alanine—Formula—C₃H₇NO₂—Alanine is a non-essential amino acid since the body readily produces it. One main dietary source is in meats. Alanine is an important amino acid in collagen and other similar proteins. Its use in the compositions is to enhance the ability to produce collagen.

Aloe Vera Gel—Aloe barbadensis mil—It is a short growing plant. Studies have not confirmed its use for treating burns, wounds, and sunburn. Topical aloe vera is not associated with toxicity issues or side effects. Historically, aloe vera was used topically and continues use in traditional medicine today. Its role in the composition relates to a potential role in healing cutaneous damages due to different causes and to increase the rate of healing.

Arginine (ARG)—Formula—C₆H₁₄N₄O₂— An α-amino acid that is used in the biosynthesis of proteins. In humans, arginine is classified as a semiessential or conditionally essential amino acid, depending on the developmental stage and health status of the individual.

Artic Fish Collagen—Arctogadus glacialis—The arctic cod lives in the arctic seas at cold temperatures and is thus one of the few species whose collagen is a liquid at cold and room temperatures. Collagen from non-arctic animals and non-arctic fish is a solid at room temperature. For the compositions, dissolvable collagen is a major advantage in utilizing the protein in the compositions that is in a liquid form rather than a solid form so it can readily react with the other components. Its use in the compositions is to provide collagen for wound support and softening.

Benzocaine—Formula—C₉H₁₁NO₂—Benzocaine is a local or topical anesthetic to relieve topical pain. It appears in many compositions to treat a variety of pain related problems. Local anesthesia for oral and pharyngeal mucous membranes is common for sore throats, mouth ulcers, denture irritation, earache, insect bites, and similar topical irritations. Few side effects are noted as long as intended doses are utilized. Its addition in the compositions is only applicable to the two types of oral mucosal lesions described and should not be given to young children.

Distilled water—Formula—H₂O—It is water that has been boiled into vapor and condensed back into liquid in a separate container. Impurities in the original water that do not boil below or at the boiling point of water remain in the original container. Distilled water can also refer to reverse osmosis and ultrafiltration of tap water. Thus, distilled water is purified water.

Ferulic Acid—Formula—C₁₀H₁₀O₄— Ferulic acid is a hydroxycinnamic acid and a natural phenol that is a major anti-oxidant that readily reacts with free radicals that accelerate aging of the skin. Its use in the compositions to reduce free-radical damage at the time of attempting to stimulate new cell growth and metabolism during the use of the compositions.

Glutamine (GLU)—Formula—C₅H₁₀N₂O₃— An α-amino acid that is used in the biosynthesis of proteins. Its side chain is similar to that of glutamic acid, except the carboxylic acid group is replaced by an amide.

Glycine—Formula—C₂H₅NO₂—Glycine is the simplest possible formula for an amino acid and remains in dissolved in both hydrophobic or hydrophilic solutions. Its use in the compositions is to enhance the ability to form collagen.

Histidine (HST)—Formula—C₆H₉N₃O₂— An α-amino acid and is a positively charged amino acid at physiological pH. The histidine amino acid is a precursor for histamine, an amine produced in the body necessary for inflammation.

Hydroxyproline (HDP)—Formula—C₅H₉NO₃— A common non-proteinogenic amino acid. Hydroxyproline is a major component of the protein collagen, comprising roughly 13.5% of mammalian collagen. Hydroxyproline and proline play key roles for collagen stability. They permit the sharp twisting of the collagen helix. Hydroxyproline is found in few proteins other than collagen.

Isoleucine (ISL)—Formula—C₆H₁₃NO₂— An α-amino acid and must be ingested in the diet since it is essential in humans.

Lecithin—Lecithin is a generic term including animal and plant materials that are both hydrophilic and lipophilic mixtures of glycerophospholipids, that include phosphatidylcholine, phosphatidylethanolamine, phosphatidylinositol, and phosphoric acid. It can be processed from soybeans, eggs, milk, marine sources, rapeseed, cottonseed, and sunflower. While it has low solubility in water, it is an excellent emulsifier. Its use in the compositions is to function as an emulsifier and dispersing agent for the combination of hydrophilic and hydrophobic components.

Leucine (LUC)—Formula—C₆H₁₃NO₂— An essential amino acid. Leucine exhibits pharmacological activity in humans and have been demonstrated to promote protein biosynthesis. Leucine is a dietary amino acid with the capacity to directly stimulate myofibrillar muscle protein synthesis.

Linoleic Acid (LOA)—Formula—C₁₈H₃₂O₂— A polyunsaturated omega-6 fatty acid, an 18-carbon chain with two double bonds in cis-configuration. It typically occurs in nature as a triglyceride ester. Free fatty acids are typically low in foods. Linoleic acid belongs to one of the two essential fatty acids.

Lysine (LYS)—Formula—C₆H₁₄N₂O₂— An α-amino acid and is essential in humans. Lysine plays a role in crosslinking of collagen polypeptides, uptake of essential mineral nutrients, and in the production of carnitine. Due to its importance in several biological processes, a lack of lysine can lead to several disease states including defective connective tissues, impaired fatty acid metabolism, anemia, and systemic protein-energy deficiency.

Methyl Sulfonyl Methane—Formula—C₂H₆O₂S—Methyl Sulfonyl Methane (MSM) has one additional oxygen compared to the similar Dimethyl Sulfoxide (DMSO). It is found naturally in a number of primitive plants and in the atmosphere above marine areas. It has been tested in a number of medical conditions similar to DMSO, but does not have any medical disease approval by the FDA. It is sold as a dietary supplement without any substantiated claims. For cutaneous application, its primary role may simply be enhancement of compositions through the skin rather than a primary effect by itself. Although, it may have anti-inflammatory effects as does DMSO. The FDA has approved MSM for the Generally Regarded as Safe (GRAS) status. A number of animal studies suggest MSM benefits in oxidative stress and inflammation, but not sufficient for drug approval. MSM primary use in the compositions is to enhance cutaneous transfer of agents through the skin. It may also add relief as an anti-oxidant and an anti-inflammatory.

Niacinamide—Formula—C₆H₆N₂O—Niacinamide or nicotinamide are like niacin, critical vitamins to a number of important functions, including the NAD/NADP complex. NAD is critical to catabolism of fat, carbohydrate, protein, and alcohol and cell signaling and DNA repair. NADP is important for anabolic reactions in fatty acid and cholesterol synthesis. Its use in the compositions along with niacin is to provide these required compounds that are important in these types of reactions.

Oleic Acid (OLA)—Formula—CH₃(CH₂)₇CH═CH(CH₂)₇COOH—A fatty acid that occurs naturally in various animal and vegetable fats and oils. It is an odorless, colorless oil. In chemical terms, oleic acid is classified as a monounsaturated omega-9 fatty acid. It is the most common fatty acid in nature.

Proline—Formula—C₅H₉NO₂—Proline is a non-essential amino acid that the body can produce. Proline is biosynthetically derived from the amino acid L-glutamate. However, its commercial synthesis is from diethyl maionate and acrylonitrile. It is used in the compositions to enhance the production of collagen.

Protocatechuic Acid (PCT)—Formula—C₇H₆O₄— A dihydroxybenzoic acid which is a type of phenolic acid. Protocatechuic acid is antioxidant and anti-inflammatory.

Sericin—Formula—C₃₀H₄₀N₁₀O₁₆—It is a protein created by Bombyx mori (silkworms) in the production of silk. Silk consists of 70-80% fibroin and 20-30% sericin. Fibroin is the structural center of the silk and sericin being the gum coating the fibers and allowing them to stick to each other. Composed structurally of 18 different amino acids, and 32% serine in a randomized amorphous coil. Sericin can be easily converted into a β-sheet conformation, via repeated moisture absorption and mechanical stretching. Sericin has been used in medicine and cosmetics. Sericin is primarily used in medicine for wound suturing due to its elasticity, tensile strength, and a natural affinity for keratin. It has a natural infection resistance and biocompatibility, and can be used as a wound coagulant. When used in cosmetics, sericin has been found to improve skin elasticity and several anti-aging factors which include an anti-wrinkle property. This is done by minimizing water loss from the skin.

Sodium Polyacrylate—Formula—[—CH₂—CH(CO₂Na)—]ₙ—It is a sodium salt of polyacrylic acid is an anionic polyelectrolyte with negatively charged carboxylic groups in the main chain and has a broad application in consumer compositions. This super absorbent polymer has the ability to absorb as much as 100 to 1000 times its mass in water.

Vitamin A (VTA)—A group of unsaturated nutritional organic compounds that includes retinol, retinal, retinoic acid, and several provitamin A carotenoids (most notably beta-carotene). Vitamin A has multiple functions. It is important for growth and development, maintenance of the immune system and good vision. Vitamin A is needed by the retina of the eye in the form of retinal. Vitamin A also functions in a very different role as retinoic acid (an irreversibly oxidized form of retinol), which is an important hormone-like growth factor for epithelial and other cells.

Vitamin B3 (VTB3) or Niacin—Formula—C₆H₅NO₂—Niacin cannot be directly converted to niacinamide, but both are critical precursors of the important NAD/NADP complex. NAD is critical to catabolism of fat, carbohydrate, protein, alcohol, cell signaling and DNA repair. NADP is important for anabolic reactions in fatty acid and cholesterol synthesis. Niacin also is a vasodilator that can also aid in the absorption of the composition on the skin. Their uses in the compositions along with niacinamide are to provide these required compounds that are important in these reactions.

Vitamin C—Formula—C₆H₈O₆—Vitamin C or Ascorbic Acid is water soluble and found in dairy and food supplies. Vitamin C is an essential nutrient that is required for tissue repair and a cofactor in multiple enzymatic reactions. Vitamin C is critical for collagen production, carnitine production, and neurotransmitter production. It functions importantly in the body as an anti-oxidant protecting against oxidative stress. It is used in the compositions as an anti-oxidant, for tissue repair, and collagen production. An alternative to Vitamin C is D-iso-ascorbic acid that blocks collagen formation, opposite to Vitamin C. It is only used in the one composition as a topical use in cutaneous Scleroderma to attempt to limit the excessive collagen production that causes skin thickening and joint movement limitations. It is the excessive collagen formation in the subcutaneous site that builds up as a response in Scleroderma that is stimulated by its inflammation and the responses to that inflammation.

### Additional Beneficial Components of Compositions

Emu Oil—Dromaius novaehollandiae is the emu that is indigenous to Australia. The oil is extracted from adipose tissue with this industry refined product containing 70% unsaturated fatty acids. Oleic acid, a mono-unsaturated omega-9-fatty acid, is the largest component. It also contains 20% linoleic acid, an omega-6-fatty acid and 2% linoleic acid, an omega-3-fatty acid. Its claims as a dietary supplement have not been confirmed. However, recent clinical studies suggest efficacy in its use as a skin moisturizer and as an insect repellent. Its proposed use in the product is as a skin moisturizer.

Emu Oil Solids—Dromaius novaehollandiae is the emu that is indigenous to Australia. After the oil is extracted from processed adipose tissue, there is a residual oil based, semi-solid product that can be used as a thickener for skin care products. It is used in the product as a thickener replacement for bees wax.

Helichrysum Italicum Essential Oil—Helichrysum Italicum is one of 600 species of Helichrysum with its oil designated as an essential oil. It is part of the sunflower family occurring in South Africa, Madagascar, Australasia, and Euroasia. Its primary use in the formulation is as a standard fragrance.

Hyssop Oil—Hyssopus officinalis is from southern Europe and the Middle East commonly used as a medicinal plant, predominantly as an antiseptic, expectorant, and cough depressant that can also stimulate the gastrointestinal system. Its use in the product is as an oil with an antiseptic nature. It is limited to products for use only in adults as it can cause seizures in young children.

Lavender Essential Oil—Lavendula augustiflora is a species of lavender from which the oil is produced that is a complex mixture of natural phytochemicals with the major components being linalool and linalyl acetae and the major supply produced in Bulgaria. It has a long usage in perfumes and aromatherapy. Its primary contribution to the product is fragrance.

Patchouli Essential Oil—Pogostemon patchouli or P cabin are shrub-like plants originally in tropical regions of Asia, but are now cultivated widely in tropical areas due to the popular nature of this fragrance. This essential oil is prepared by steam distillation of the leaves. Three primary components of this oil are Patchoulol—Formula—C₁₅H₂₆O—that is the primary essence of the scent of the product, Norpatchoulenol—Formula—C₁₄H₂₂O—that is a terpenoid component that also effects the primary scent, and Germacrene—C₁₅H₂₄— that has anti-microbial properties. This essential oil has been used in perfumes for centuries and is now found in insect repellents and alternative medicines. Its use in the product is primary for fragrance but also for its anti-microbial component.

Ylang-Ylang Essential Oil—Cananga odorata, known as the tropical cananga tree in Indonesia, Malaysia, and the Philippines, is valued for its Ylang-Ylang flowers that produce a popular oil through steam distillation that has been used for centuries in perfume. Its primary components are Linalool, Germacrene, Caryophyllene, p-cresyl methyl ether, methyl benzoate, and sesquiterpenes. Its proposed medicinal usages include reduction of blood pressure, reduction of sebum secretion in the skin, and as an aphrodisiac. Its primary use in the product is as a fragrance, although it contains several components that are in other valuable oil products from this region that we use.

Berberine HCL—Formula—C₂₀H₁₈NO₄—Berberis vulgaris is one of several plants with berberine as a quarternary ammonium salt with a very strong yellow color used for dying wool and other fabrics. It was reported in use in China as a folk medicine in 3,000 BC. It currently is a research product for different potential treatments of arrhythmia, diabetes, hyperlipidemia, and cancer. Berberine is considered an antibiotic and is being evaluated as a treatment for methicillin-resistant Staph infections. It is used in the product as a potential anti-microbial as well as a marker for the lack of anti-oxidant activity that would reduce the yellow color.

Chlorhexidine—Formula—C₂₂H₃₀Cl₂N₁₀—Chlorhexidine is used in disinfectants, cosmetics, and pharmaceutical products as an anti-microbial agent that appears more potent than provodone-iodine (Betadine). It is active against Gram-positive and Gram-negative organisms, facultative anaerobes, aerobes, and yeasts. It is particularly effective against Gram-positive bacteria (≥1 μg/l). Significantly higher concentrations (10 to 73 μg/ml) are required for Gram-negative bacteria and fungi. At physiologic pH, it disassociates and releases the positively charged chlorhexadine cation that readily binds to the negatively charged bacterial cell wall. At low doses, its membrane binding acts as bacterial static effect. At high doses, its binding to the bacterial cell wall causes cell wall disruption and destruction of the microbe. It is on the World Health Organization's List of Essential Medicines. It is used in the product for its potent anti-microbial activities.

Natamax—Formula—C₃₃H₄₇NO₁₃—Natamycin (Natamax) is a naturally occurring anti-fungal agent produced during fermentation by Streptomyces natalensis that is commonly found in soil. It is used in the food industry as a natural preservative. It is listed on the World Health Organization's List of Essential Medicines. It has been used in the food industry for decades in dairy products and other foods to retard microbial growth. Its medical use is usually in the form of a cream, lozenge, or drops for the eye or ear. It does not have any acute toxicity in animals or humans. It inhibits fungal growth by inhibiting amino acids and glucose passage across plasma membranes. It is used in the product to inhibit fungal growth.

Panthenol—Formula—C₉H₁₉NO₄— Panthenol is an alcohol analogue of Vitamin B5 or pantothenic acid that is commonly utilized in pharmaceuticals, cosmetics, and skin care products as a moisturizer and humectant. It readily penetrates the skin and mucous membranes where it is converted to pantothenic acid that readily binds water and enables reactivity with the other product components and with the tissues. It is odorless and colorless. Its inclusion in the product is for its moisturizing and humectant qualities.

Quercitin—Formula—C₁₅H₁₀O₇—Quercitin is a plant polyphenol in the flavonoid group that is widely found in many vegetables and in many supplements. It is a form of many flavonoid glycosides. In spite of many studies, there is no clear evidence that the use of quercitin is useful in the treatment of cancer or any other diseases. This may in part be due to the fact that quercitin has a very short half-life in the human body. The question for the product is if its half-life can be longer in these topical to subcutaneous products. Its in vitro pharmacology supports the anti-oxidant activities of this compound. Its use in the product is to be an anti-oxidant in the skin and subcutaneous sites.

Ubiquitin—Formula—C₈₉H₁₅₁N₂₇O₂₄— Ubiquitin is a small, regulatory protein that exists in all eukaryotic cells, those with a nucleus and membrane bound organelles. It has 4 genes in the human genome that code for it. Ubiquitin carries out a multitude of functions predominantly through protein conjugations that result in many different protein modifications, from induced degradation, to change cellular location, to alter their activities, and to either promote or inhibit protein interactions. They can also form chains together to add another dimension to their protein interactions. Ubiquitin is highly conserved with human cells and yeast sharing 96% sequence identity. Abnormal ubiquitin activity is associated with many different diseases that can have tissue sections staining for excessive accumulations of ubiquitin based compounds including Alzheimer's (neurofibrillary tangles), Parkinson's (Lewy body), Huntington's (inclusions in motor neurons), alcoholic liver disease (Mallory bodies), and brain astrocytes (Rosenthal fibers). A number of studies have related that defects and alterations in ubiquination processes are often found in a variety of cancers, including renal cell, breast, cervical, colorectal, and glioblastoma. Ubiquitin is utilized in the product to potentially promote normal cell replication and growth, both intimately involved with the product.

Vanzan—Formula—C₁₃H₁₀O—Zanthan gum, a polysaccharide, that was discovered as a product of fermentation from a strain of bacteria, Xanthomohas campestris, that can be commercially produced using these bacteria given simple sugars and the proper fermentation conditions. It has many uses regarding its ability to readily increase viscosity in a variety of liquids, including in foods and drinks. It is used in the product to increase thickening as required above that achieved by phase change and other agents.

### Groups of Components

### Core

### α-Pinene (APN)

### Glycerol (GLY)

### Ghana Shea Butter (GSB)

### Black Cumin Seed Oil (BCS)

### AF (Artic Fish) Collagen (AFC)

### Aloe Vera Leaf Gel (AVLG)

### Lecithin (LCT)

### Methyl Sulfonyl Methane (MSM)

### Functional Oil

### Apricot Kernal Oil (AKO)

### Argan Nut Oil (AGN)

### Baobab Oil (BAB)

### Calendula Oil (CDA)

### Camellia Oil (CMA)

### Caprylic, Capric Triglyceride (CCT)

### Caryophyllene (CAP)

### Elemi Oil (EMO)

### Kanuka Oil (KNO)

### Litsea Cubeba Oil (LCO)

### Marula Oil (MRL)

### Palmarosa Oil (PLO)

### Palo Santo

### Rose Hip Seed oil

### Sachi Inchi oil

### Sea Buckthorn Oil (SBO)

### Tamanu Oil (TMU)

### Alcohol

### Benzyl Alcohol (BA)

### Cetyl Alcohol (CTA)

### Panthenol (PNT)

### Anti-Inflammatory

### Acai Berry

### Chamomile German Oil (CGB)

### Capaiba Oil (CPO)

### Myrrh

### Rose Hip Seed Oil

### Oleic Acid (OLA)

### Linoleic Acid (LOA)

### Anti-Oxidant

### Kaempferol

Caffeic acid

### Protocatechuic Acid (PCT)

### Anti-Microbial

### Anethole Anise Camphor

### Cabreuva Oil

### Carvacrol (CVC)

### Niaouli Oil

### Thyme Red, White

### Winter Savory

### Floral

### Rosemary Oil (RMO)

### Thickener

### Benzoin (BZN)

### Wax

### Bees Wax (BW)

### Flavors

Cedarwood essential Oil

### Fragrance

### Carvone Oil (CRV)

### Geranium Oil (GER)

### Ho Wood Oil (HWO)

### Jasmine Oil (JAS)

### Lavandin Oil (LVO)

### Limonene Oil (LMN)

### Nerolina Oil (NRL)

### Peppermint

### Terpineol

### Vetiver

### Fatty Acid

### Ferulic Acid (FRA)

### Analgesic

### Benzocaine

### Amino Acid

### Alanine (ALA)

### Arginine (ARG)

### Glutamine (GLU)

### Glycine (GLY)

### Histidine (HST)

### Hydroxyproline (HDP)

### Isoleucine (ISL)

### Leucine (LUC)

### Lysine (LYS)

### Proline (PRL)

### Carbohydrate

Agave nectar (AGV)

### Vitamin

### Vitamin A (VTA)

### Vitamin B3 (VTB3) or Niacin

### Vitamin C (VTC)

### Vitamin E (VTE)

### Niacinamide (NCM)

### Solvent

Distilled water

### Gel

### Sericin

### Moisturizer

### Sodium Polyacrylate

### 1-Tetradecanol

### EXAMPLES

### Dupytren's Disease and Contracture

### Example 1

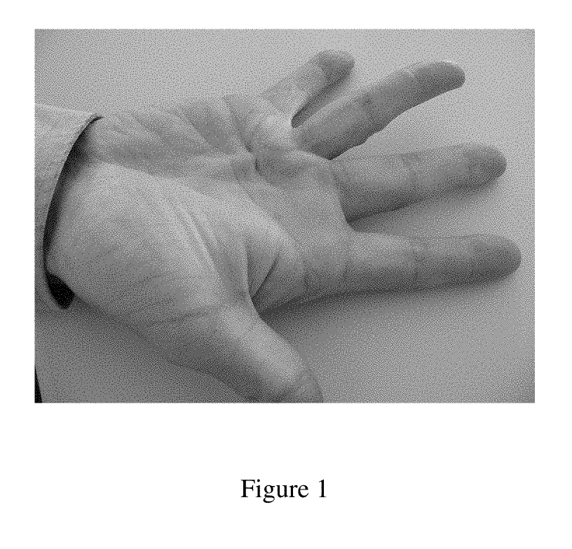

Dupytren's Disease of the hands is an early form of what most likely will evolve overtime into Dupytren's Contracture of the hands. This relatively infrequent disease is based on northern European ancestry that increases in incidence typically from age 45-50 years of age with increasing incidence and severity of existing disease throughout the rest of these people's lives. There is a mild increase in the incidence of males over females in the progression of the disease. The earlier in life it begins the expectation of more accelerated progression and the involvement of more fingers will be experienced. It converts from Dupuytren's Disease to Dupuytren's Contracture when the fingers begin to bend in towards the palms. The abnormality begins as the Disease with the discovery of a subcutaneous nodule on the palmar surface of the hand that is usually not tender at the base of fingers 3, 4, and 5 on either hand. After a relatively short time, abnormal skin folds begin to develop below the nodule into the palm. Additional nodules may form then or at any time in the future on either hand mostly involving fingers 3, 4, and 5. Another example of involvement of any finger is observed by placing the palm on a flat surface and determining the ability to raise the affected finger up off the table. Over time, the involved fingers will not be able to rise above the flat surface. Progression of this problem is expected for any involved finger in Dupytren's Disease to Dupytren's contracture whenever the angle between any finger and the palm begins to decrease from vertical with the hands held upright. Ongoing disease progression of Dupytren's Contracture will be noted with further movement of the fingers into the palm, as shown in FIG. 1. This degree of curvature is measured to document ongoing progression of the disease.

The disclosed composition halts the progression of Dupuytren's disease to the Dupuytren's contracture stage. All nodules show significant decrease in size which appears to happen within the first 2-4 months of treatment.

### Example 2

White male, 69 years old, developed palm nodules and skin folds at base of both left and right hands with rigid third digits in 2014. When the left hand had progressed to bend towards the palm at 5 degrees in 2015, the first preliminary product formulation used twice daily, thoroughly rubbing it into the skin, made nodules disappear and stopped progression of contracture of third digit. The composition was applied to both fingers twice a day. Within a few weeks of using the composition, the nodule size and thickness began to decrease, first on the left hand and then followed on the right hand. More importantly, the left fourth finger had returned back to vertical, reversing from the early Contracture Stage back to the Disease Stage of Dupuytren's. Many months usage of the product kept these progressions limited. However, when no product was available for 6 months in 2017, ventral surface cords developed with new chords extending into the palm on both the left and right hands extending from the fourth fingers down into the palm. When product became available again, the previous progression of these lesions halted with continuous usage of this product twice a day up to November, 2018. Current observations show decreased skin folds, softening of the flexor chords, and no new contractures noted with continued twice a day usage.

### Example 3

White male, 66 years of age developed skin nodules and skin folds on left and right third digits at the base. Both nodules disappeared and skin folds decreased with 6 months of twice a day applications.

### Example 4

A small clinical trial for the use of the disclosed composition for patients with active Dupuytren's Disease and Contracture was conducted in Ukraine with two patients.

The first is a 73 year old male, 5 foot 6 inches weighing 139 pounds who had untreated contractures on the right hand for 11 years and on the left hand for 16 years. Prior to treatment, the right hand had a fifth finger Stage 3 contracture with 100° of flexion and a left hand fifth finger Stage 4 contracture with 150° of flexion. After 6 weeks of treatment with the disclosed composition, the right hand fifth finger contraction reduced to a Stage 2 contracture with 55° of flexion, but the left hand Stage 4 contraction treated for the same time remained a stage 4 lesion that only reduced to a 140° of flexion.

The second patient in this preliminary trial was also a 73 year old male, 5 foot 8 inches weighing 194 pounds who had untreated contractures on the right fifth digit Stage 1 with 25° of flexion and a fourth digit Stage 2 with 75° of flexion for 13 years. The left hand had a fifth digit Stage 3 contraction with 95° of flexion and a fourth finger Stage 4 contraction with 140° contraction for 9 years. After 6 weeks of treatment with the disclosed composition, the right hand fith digit Stage 1 lesion with 25° of flexion remained a Stage 1 but no longer had any degrees of flexion. The right hand fourth digit Stage 2 contracture with 75° flexion reduced to 55° of flexion. The left hand fifth digit Stage 3 contracture with 95° of flexion reduced to a Stage 2 contraction with 45° of flexion. The left hand fourth finger Stage 4 contraction with 140° of flexion reduced to a Stage 3 contracture with 110° of contraction.

Table 5 lists the composition formulation for treatment of Dupuytren's Disease and Contracture.

TABLE 5
Dupuytren's Disease and Contracture Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Apricot Kemal Oil (AKO) | 5 | ml | AF (Artic Fish) Collagen (AFC) | 40 | g |
| Argan Nut Oil (AGN) | 5 | ml | Agave nectar (AGV) | 10 | ml |
| Baobab Oil (BAB) | 2.5 | ml | Alanine (ALA) | 5 | g |
| Bees Wax (BW) | 40 | g | Aloe Vera Leaf Gel (AVLG) | 275 | g |
| Benzoin (BZN) | 4 | ml | Arginine (ARG) | 5 | g |
| Benzyl Alcohol (BA) | 50 | ml | Distilled water | 500 | ml |
| Calendula Oil (CDA) | 5 | ml | Ferulic Acid (FRA) | 10 | g |
| Camellia Oil (CMA) | 10 | ml | Glutamine (GLU) | 5 | g |
| Capaiba Oil (CPO) | 10 | ml | Glycine (GLY) | 5 | g |
| Caprylic, Capric Triglyceride (CCT) | 10 | ml | Histidine (HST) | 5 | g |
| Carvacrol (CVC) | 5 | ml | Hydroxyproline (HDP) | 5 | g |
| Caryophyllene (CAP) | 10 | ml | Isoleucine (ISL) | 5 | g |
| Cetyl Alcohol (CTA) | 40 | g | Lecithin (LCT) | 25 | g |
| Chamomile German Oil (CGB) | 10 | ml | Leucine (LUC) | 5 | g |
| Elemi Oil (EMO) | 5 | ml | Linoleic Acid (LOA) | 5 | ml |
| Geranium Oil (GER) | 2.5 | ml | Lysine (LYS) | 5 | g |
| Ghana Shea Butter (GSB) | 275 | g | Methyl Sulfonyl Methane (MSM) | 25 | g |
| Glycerol (GLY) | 10 | ml | Niacinamide (NCM) | 2.5 | g |
| Ho Wood Oil (HWO) | 2.5 | ml | Oleic Acid (OLA) | 5 | ml |
| Jasmine Oil (JAS) | 2.5 | ml | Proline (PRL) | 5 | g |
| Kanuka Oil (KNO) | 2.5 | ml | Protocatechuic Acid (PCT) | 10 | g |
| Lavandin Oil (LVO) | 10 | ml | Sericin | 50 | g |
| Limonene Oil (LMN) | 40 | ml | Sodium Polyacrylate | 20 | g |
| Litsea Cubeba Oil (LCO) | 5 | ml | Vitamin A (VTA) | 2.5 | g |
| Marula Oil (MRL) | 2.5 | ml | Vitamin B3 (VTB3) or Niacin | 12.5 | g |
| Nerolina Oil (NRL) | 2.5 | ml | Vitamin C (VTC) | 2.5 | g |
| Black Cumin Seed Oil (BCS) | 50 | ml | Vitamin E (VTE) | 2.5 | g |
| Palmarosa Oil (PLO) | 2.5 | ml | | | |
| Panthenol (PNT) | 10 | ml | | | |
| Rosemary Oil (RMO) | 5 | ml | | | |
| Sea Buckthorn Oil (SBO) | 5 | ml | | | |
| Tamanu Oil (TMU) | 5 | ml | | | |

### Abrasions, Cuts, & Minor Burns

### Example 5

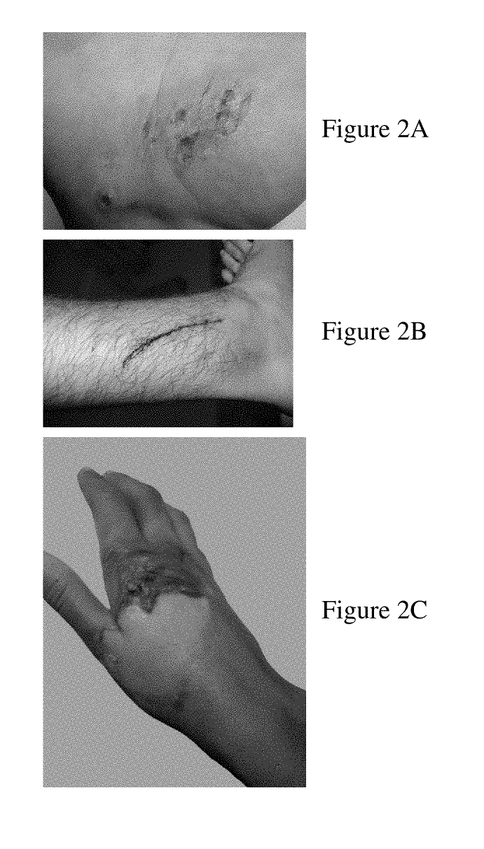

There are many first aid products currently on the market focused on treating abrasions (FIG. 2A), cuts (FIG. 2B), and minor burns (FIG. 2C) that have a variety of effectiveness and varied times to achieve healing compared to no treatment besides cleansing. One of the oldest compositions and most successful in terms of global distribution is a triple antibiotic ointment called NEOSPORIN™ by Johnson & Johnson (1952). Its formulation is very basic including three antibiotics: neomycin, polymyxin, and bacitracin. It also contains petroleum jelly, cocoa butter, cottonseed oil, sodium pyruvate, and tocopheryl acetate. Since many users develop allergies to neomycin, Johnson & Johnson offers a double antibiotic composition containing only bacitracin and polymyxin called POLYSPORIN™. The NEOSPORIN™ composition, while very popular for years, has essentially no ingredients proven to hasten healing. Petroleum jelly from oil compositions is also called Vaseline as branded by Unilever and only prevents moisture loss from the skin. The only potentially healing agent would be cocoa butter that is processed from whole cocoa beans and melts at just under body temperature, making it convenient to use in many compositions, including soaps and lotions. Cocoa butter contains Oleic acid (34.5%—Omega-9, used in food and soap), Stearic acid (34.5%—surfactant, detergents, soaps, lubricants), Palmitic acid (26.0%—soaps, food compositions, cosmetics), Linoleic acid (3.2% —Omega-6, essential oil, surfactant, anti-inflammatory), Arachidic acid (1.0%—detergents, lubricants) and Palmitoleic acid (0.3% —Omega-7). Cottonseed oil is a cooking oil. Sodium pyruvate protects against peroxides. Tocopherol acetate is a form of Vitamin E that can resist acid destruction. None of these NEOSPORIN™ components are able to hasten healing in the skin.

Establishing formulations for these problems is complicated by utilizing many of the components collected due to their long history of enabling accelerated healing. The disclosed composition has been used by nearly a dozen people who had received abrasions, cuts, and minor burns at different times. The results of their use of the composition show a consistent ability to heal these skin wounds rapidly, usually within 2 to 7 days depending upon the size of the wound. Small wounds or injuries usually are sealed and healed within 2-4 days. The larger abrasions on extremities heal well with little evidence of scarring within 7-9 days that would normally take 10 to 14 days. So these results demonstrate a composition that clearly improves healing in a short time scale with little scarring after it is healed. There also is a clear protection against microbial contamination and infection at the same time. In fact, one can simply apply the composition to an infected finger nail or toe nail that had that had been previously cut or injured that over a few days will show the infection has been eliminated while the composition continues to hasten healing.

### Example 6

A 39 year white male was a bicycle rider going on trails in the area. He fell while riding a trail and landed on his left side inducing a large, 5 by 12 cm skin abrasion with bleeding. On return from the ride, he washed it off and placed NEOSPORIN™ on the wound. The next day, he was offered to replace the NEOSPORIN™ with the disclosed composition. His experience with NEOSPORIN™ for this kind of partial thickness skin wound, would take 10 to 14 days for it to heal. The three times a day usage of this product permitted the healing to be essentially over in 5-6 days with little evidence of scarring.

### Example 7

A 70 year old white male fell at home while working outside and suffered a superficial laceration of the skin on the knee. The disclosed composition was applied to the injury after cleaning. The disclosed composition was applied three times a day. By the second day, the wound was closed and no longer weeping fluid and had lost most of its pain. By the fourth day, the wound was essentially healed over and without pain or sign of infection.

### Example 8

A 52 year old white male applied the disclosed composition on multiple occasions of scratches, scrapes, and cuts. The disclosed composition treats injuries without infection, and healed in half the time usually required for NEOSPORIN™.

Table 6 lists the composition formulation for treatment of cuts, abrasions, and minor burns.

TABLE 6
Abrasions, Cuts, and Minor Burns Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Acai Berry | 10 | ml | AF (Artic Fish) Collagen (AFC) | 40 | g |
| Anethole Anise Camphor | 5 | ml | Agave nectar (AGV) | 10 | ml |
| Argan Nut Oil (AGN) | 5 | ml | Alanine (ALA) | 5 | g |
| Baobab Oil (BAB) | 5 | ml | Aloe Vera Leaf Gel (AVLG) | 275 | g |
| Bees Wax (BW) | 40 | g | Arginine (ARG) | 5 | g |
| Benzoin (BZN) | 4 | ml | Distilled water | 500 | ml |
| Benzyl Alcohol (BA) | 50 | ml | Ferulic Acid (FRA) | 10 | g |
| Cabreuva Oil | 5 | ml | Glutamine (GLU) | 5 | g |
| Calendula Oil (CDA) | 5 | ml | Glycine (GLY) | 5 | g |
| Camellia Oil (CMA) | 10 | ml | Histidine (HST) | 5 | g |
| Capaiba Oil (CPO) | 10 | ml | Hydroxyproline (HDP) | 5 | g |
| Caprylic acid (CCT) | 10 | ml | Isoleucine (ISL) | 5 | g |
| Carvacrol (CVC) | 5 | ml | Lecithin (LCT) | 25 | g |
| Caryophyllene (CAP) | 10 | ml | Leucine (LUC) | 5 | g |
| Cetyl Alcohol (CTA) | 40 | g | Linoleic Acid (LOA) | 5 | ml |
| Chamomile German Oil (CGB) | 10 | ml | Lysine (LYS) | 5 | g |
| Ghana Shea Butter (GSB) | 275 | g | Methyl Sulfonyl Methane (MSM) | 25 | g |
| Glycerol (GLY) | 10 | ml | Niacinamide (NCM) | 2.5 | g |
| Kaempferol | 5 | g | Oleic Acid (OLA) | 5 | ml |
| Kanuka Oil (KNO) | 2.5 | ml | Proline (PRL) | 5 | g |
| Limonene Oil (LMN) | 40 | ml | Protocatechuic Acid (PCT) | 10 | g |
| Litsea Cubeba Oil (LCO) | 5 | ml | Sericin | 50 | g |
| Myrrh | 5 | ml | Sodium Polyacrylate | 20 | g |
| Nerolina Oil (NRL) | 2.5 | ml | Vitamin A (VTA) | 2.5 | g |
| Niaouli Oil | 5 | ml | Vitamin B3 (VTB3) or Niacin | 12.5 | g |
| Black Cumin Seed Oil (BCS) | 50 | ml | Vitamin C (VTC) | 2.5 | g |
| Panthenol (PNT) | 10 | ml | Vitamin E (VTE) | 2.5 | g |
| Peppermint | 5 | ml | | | |
| Rosemary Oil (RMO) | 5 | ml | | | |
| Sachi Inchi oil | 5 | ml | | | |
| Sea Buckthorn Oil (SBO) | 5 | ml | | | |
| Tamanu Oil (TMU) | 5 | ml | | | |
| Terpineol | 5 | ml | | | |
| Thyme Red, White | 5 | ml | | | |

### Peyronie's Disease or Induratio Penis Plastica (IPP)

### Example 9

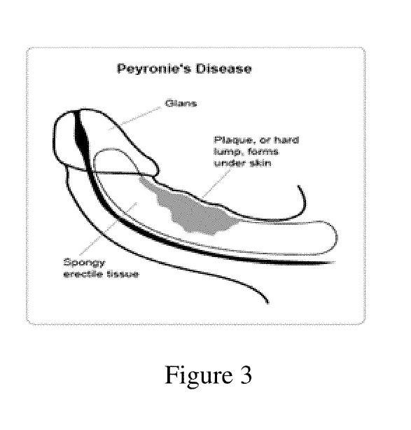

Peyronie's Disease is a connective tissue disorder of the penis affecting 5% of the male population. It involves fibrous plaques developing along the fibrous sheath, the tunica albuginea, that surrounds the spongy erectile tissue, the corpus cavernosa, in the penis. When the penis fills with blood and erects, these fibrous plaques or bands prevent that portion of the penis from erecting properly. This leads to two types of deformities of the penis: the more common is a marked curvature of the penis in any direction at the point of the stricture (FIG. 3) while the least common is an hour glass stricture completely around the penis. Regardless of the type of stricture, as it progresses it causes more deformity. Penile penetration during intercourse becomes painful and eventually impossible. A small percentage of men with Dupuytren's Disease or Contracture also develop Peyronie's Disease for unknown reasons. There is no known cause of these progressive deformities of the penis. Without treatment, 10-15% will spontaneously improve over time, 40-50% will get worse, and 35-50% will not change. Currently, there is no effective treatment that does not include a high risk of doing more harm. Injection of collagenase into the plaques can soften them to reduce the strictures causing the deformities. However, if the physician happens to inject collagenase into the spongy erectile tissue, it can be weakened and fracture during intercourse producing a non-functional penis. Trying to surgically remove these plaques has a high risk of permanently injuring the penis, which can lead to internal fracture leading to non-function regarding intercourse. At this point, there is no effective treatment to this disease without a high risk of making it worse.

Due to the observations of the effect of the disclosed composition in people with Dupuytren's Disease and Contracture, a similar preparation was tried in men with untreated Peyronie's disease. Four patients were selected and provided with the topical composition to apply twice a day to the region of the penis involved. At one month follow up, the first two patients, one with a marked lateral curve and the other with an hour-glass deformity near the end of the penis returned quite pleased with their results. The marked curve patient's penis had the curvature reduced by 50% at that time permitting him to again have intercourse. The second patient with the hour-glass deformity had his stricture essentially resolve in this time leaving him to have fairly normal intercourse as well. They are continuing to use the medication. The second two patients are in treatment currently and will be returning with their one month follow up soon.

### Example 10

A 34 year old, middle eastern male with Peyronie's disease of the cicatrix, circumferential type with significant curvature of the penis restricting intercourse. Photos and measurements were made after injection of agents into the base of the penis to cause a maximal erection of the penis. The patient was given the disclosed composition for treatment of Peyronie's Disease and told to apply it twice a day to the lesion near by tissue in the penis. On 3 month follow up, the penile constricting lesion had significantly loosened with a resulting reduction of the curvature from 80 degrees down to 45 degrees which has been sufficient to permit intercourse. Additional treatment and follow up are underway.

### Example 11

A 38 year old, middle eastern male with Peyronie's disease of the dorsal ligament as a thickened nodule causing a dorsal and partially lateral curvature of the penis with significant curvature of the penis restricting intercourse. Photos and measurements were made after injection of agents into the base of the penis to cause a maximal erection of the penis. The patient was given the disclosed composition for treatment of Peyronie's Disease and told to apply it twice a day to the lesion near by tissue in the penis. After 4 months of follow up, the dorsal penile ligament lesion had softened permitting the curvature to reduce from 65 degrees down to 35 degrees permitting the ability to have intercourse again. Treatment continues with additional observation periods in follow up.

Table 7 lists the composition formulation for treatment of Peyronie's Disease or Induration Penis Plastica.

TABLE 7
Peyronie's Disease or Induration Penis Plastica Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Acai Berry | 10 | ml | AF (Artic Fish) Collagen (AFC) | 40 | g |
| Anethole Anise Camphor | 5 | ml | Agave nectar (AGV) | 10 | ml |
| Argan Nut Oil (AGN) | 5 | ml | Alanine (ALA) | 5 | g |
| Bees Wax (BW) | 40 | g | Aloe Vera Leaf Gel (AVLG) | 275 | g |
| Benzoin (BZN) | 4 | ml | Arginine (ARG) | 5 | g |
| Benzyl Alcohol (BA) | 50 | ml | Distilled water | 500 | ml |
| Calendula Oil (CDA) | 5 | ml | Ferulic Acid (FRA) | 10 | g |
| Camellia Oil (CMA) | 10 | ml | Glutamine (GLU) | 5 | g |
| Capaiba Oil (CPO) | 10 | ml | Glycine (GLY) | 5 | g |
| Caprylic, Capric Triglyceride (CCT) | 10 | ml | Histidine (HST) | 5 | g |
| Carvacrol (CVC) | 5 | ml | Hydroxyproline (HDP) | 5 | g |
| Caryophyllene (CAP) | 10 | ml | Isoleucine (ISL) | 5 | g |
| Cetyl Alcohol (CTA) | 40 | g | Lecithin (LCT) | 25 | g |
| Chamomile German Oil (CGB) | 10 | ml | Leucine (LUC) | 5 | g |
| Elemi Oil (EMO) | 5 | ml | Linoleic Acid (LOA) | 5 | ml |
| Ghana Shea Butter (GSB) | 275 | g | Lysine (LYS) | 5 | g |
| Glycerol (GLY) | 10 | ml | Methyl Sulfonyl Methane (MSM) | 25 | g |
| Kanuka Oil (KNO) | 2.5 | ml | Niacinamide (NCM) | 2.5 | g |
| Limonene Oil (LMN) | 40 | ml | Oleic Acid (OLA) | 5 | ml |
| Litsea Cubeba Oil (LCO) | 5 | ml | Proline (PRL) | 5 | g |
| Marula Oil (MRL) | 2.5 | ml | Protocatechuic Acid (PCT) | 10 | g |
| Black Cumin Seed Oil (BCS) | 50 | ml | Sericin | 50 | g |
| Palo Santo | 2.5 | ml | Sodium Polyacrylate | 20 | g |
| Panthenol (PNT) | 10 | ml | Vitamin A (VTA) | 2.5 | g |
| Rosemary Oil (RMO) | 5 | ml | Vitamin B3 (VTB3) or Niacin | 12.5 | g |
| Sea Buckthorn Oil (SBO) | 5 | ml | Vitamin C (VTC) | 2.5 | g |
| Terpineol | 5 | ml | Vitamin E (VTE) | 2.5 | g |

### Exposed Aging Skin

### Example 12

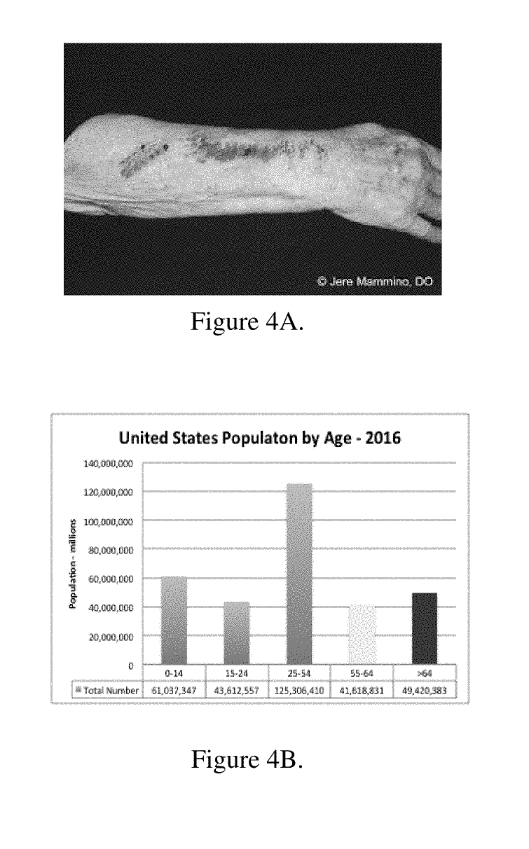

As everyone ages overtime, their skin is under easy observation, and readily shows multiple aging changes. The age relating skin changes include thinning of the outer surface of the skin, loss of substance and softness in both the outer and deeper skin components, wrinkles, easy bruising, appearance of pigmented flat lesions, scaly lesions, skin tags, loss of hair, and scratches which take much longer to heal (FIG. 4A). The appearance and the rate of the increasing incidence of these aging disorders predominantly depend upon the individual's history of sun exposure, history and the degree of routine skin care, race, as well as, genetics. An increasing incidence of skin cancers also will occur with increasing age. Routine skin care of the face, as well as, the use of sun protectants is more common in both men and women than routine skin care of the hands, arms, legs, and feet. Thus, it is very common to see multiple types of accelerated skin aging problems in the arms and hands of the vast majority of the aging populations.

If one examines the USA population by age from 2016, one finds there are 49.4 million people over the age of 64 years of age (FIG. 4B). This is 15.2% of the population that is subject to aging skin and would certainly have the skin aging readily observable and open for potential treatment. Of this age group, 44.3% are males and 55.7% are females. While aging skin can certainly begin in the 55 year olds, it is not as frequent as in the >64 year olds.

### Example 13

A 72 year old, white male, had progressive hair loss on the dorsal surfaces of both forearms and hands that had progressed over the last several years. Skin thinning had also progressed to the marked reduction of surface hair and easy bruising of the skin. Several superficial skin tears had also occurred leaving scars.

There were many actinic keratoses present on these same surfaces from previous sun damage. This person had been using moisturizers for several years on these surfaces without any effect on delaying these-age related changes. The disclosed composition was applied to the dorsal surface of the right forearm while moisturizers were continued on the left forearm. Within a couple of months, there was noticeable difference in the appearance of the two forearms. The left forearm was unchanged. The right forearm skin had begun to thicken with increased terger of the skin noted along with the epidermal layer clearly thickening. The actinic keratoses began to flatten and become thinner and smoother. Then, new hair began to appear across the right forearm without any new hair forming on the left forearm. The test subject was allowed to apply the disclosed composition to both forearms after the changes became quite obvious when examining both forearms. Several months of continued use of the disclosed composition on both forearms shows established hair, both dark and white, growing well to its normal length, increased dermal thickening, strengthening and thickening of the epidermis without any new tears, and ongoing reduction in the actinic keratoses size and appearance.

Table 8 lists the composition formulation for treatment of exposed aging skin.

TABLE 8
Old-Age Skin Treatment Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Acai Berry | 10 | ml | AF (Artic Fish) Collagen (AFC) | 40 | g |
| Argan Nut Oil (AGN) | 5 | ml | Agave nectar (AGV) | 10 | ml |
| Baobab Oil (BAB) | 5 | ml | Alanine (ALA) | 5 | g |
| Bees Wax (BW) | 40 | g | Aloe Vera Leaf Gel (AVLG) | 275 | g |
| Benzoin (BZN) | 4 | ml | Arginine (ARG) | 5 | g |
| Benzyl Alcohol (BA) | 50 | ml | Distilled water | 500 | ml |
| Cabreuva Oil | 5 | ml | Ferulic Acid (FRA) | 10 | g |
| Caffeic acid | 5 | g | Glutamine (GLU) | 5 | g |
| Calendula Oil (CDA) | 5 | ml | Glycine (GLY) | 5 | g |
| Camellia Oil (CMA) | 10 | ml | Histidine (HST) | 5 | g |
| Capaiba Oil (CPO) | 10 | ml | Hydroxyproline (HDP) | 5 | g |
| Caprylic, Capric Triglyceride (CCT) | 10 | ml | Isoleucine (ISL) | 5 | g |
| Carvacrol (CVC) | 5 | ml | Lecithin (LCT) | 25 | g |
| Caryophyllene (CAP) | 10 | ml | Leucine (LUC) | 5 | g |
| Cetyl Alcohol (CTA) | 40 | g | Linoleic Acid (LOA) | 5 | ml |
| Chamomile German Oil (CGB) | 10 | ml | Lysine (LYS) | 5 | g |
| Elemi Oil (EMO) | 5 | ml | Methyl Sulfonyl Methane (MSM) | 25 | g |
| Geranium Oil (GER) | 2.5 | ml | Niacinamide (NCM) | 2.5 | g |
| Ghana Shea Butter (GSB) | 275 | g | Oleic Acid (OLA) | 5 | ml |
| Glycerol (GLY) | 10 | ml | Proline (PRL) | 5 | g |
| Ho Wood Oil (HWO) | 2.5 | ml | Protocatechuic Acid (PCT) | 10 | g |
| Jasmine Oil (JAS) | 2.5 | ml | Sericin | 50 | g |
| Kaempferol | 5 | g | Sodium Polyacrylate | 20 | g |
| Kanuka Oil (KNO) | 2.5 | ml | Valine (VAL) | 5 | g |
| Lavandin Oil (LVO) | 10 | ml | Vitamin A (VTA) | 2.5 | g |
| Limonene Oil (LMN) | 40 | ml | Vitamin B3 (VTB3) or Niacin | 12.5 | g |
| Litsea Cubeba Oil (LCO) | 5 | ml | Vitamin C (VTC) | 2.5 | g |
| Marula Oil (MRL) | 2.5 | ml | Vitamin E (VTE) | 2.5 | g |
| Myrrh | 5 | ml | | | |
| Nerolina Oil (NRL) | 2.5 | ml | | | |
| Niaouli Oil | 5 | ml | | | |
| Black Cumin Seed Oil (BCS) | 50 | ml | | | |
| Palmarosa Oil (PLO) | 2.5 | ml | | | |
| Palo Santo | 2.5 | ml | | | |
| Panthenol (PNT) | 10 | ml | | | |
| Peppermint | 5 | ml | | | |
| Rose Hip Seed oil | 5 | ml | | | |
| Rosemary Oil (RMO) | 5 | ml | | | |
| Sachi Inchi oil | 5 | ml | | | |
| Sea Buckthorn Oil (SBO) | 5 | ml | | | |
| Tamanu Oil (TMU) | 5 | ml | | | |
| Vetiver | 2.5 | ml | | | |

### Toe Nail Fungus

### Example 14

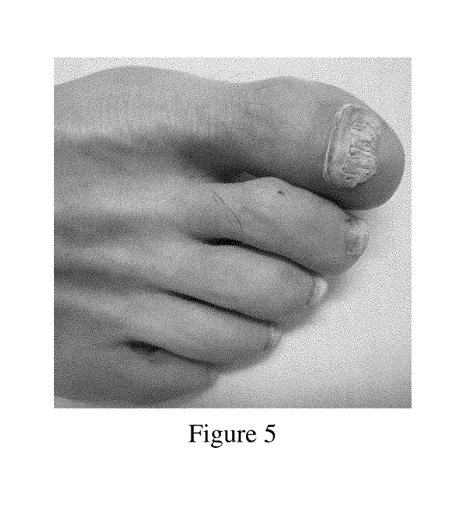

Toe nail fungus is medically known as onychomycosis, or tinea unguium, commonly affects toe nails and can be progressive to the state of nail destruction, if not treated properly (FIG. 5). It can also affect the finger nails. It is the most common of all the nail afflictions affecting 10% of the population and represents 50% of all nail afflictions. The progression is from mild forms to the most severe with loss of most of the normal nail.

The pathogens involved in onchyomycosis are all represented in the fungus kingdom that includes dermatophytes in the western world and Candida and non-dermatophyes in the tropics and subtropics. The dermatophytes most common representative is Trichophyton rubrum with multiple related species. Candida species predominantly affect the finger nails, rather than the toe nails. The non-dermatophytes are predominantly molds, mostly from the genus of Neoscytalidium, Scopulariopsis, and Aspergillus. Conditions that can be confused with onychomycosis include nail psoriasis, lichen planus, contact dermatitis, and nail bed tumors such as melanoma, thrauma, and Ω yellow nail syndrome.

Six patients with this condition have used the disclosed composition for treating infected toe nails. One patient had the most involvement with all ten toe nails involved to the severe stage. His podiatrist suggested he needed to have all ten nails removed to enable healing his problem. He was supplied with the disclosed formulation with application once a day after showering, as well as, keeping his nails trimmed well through out the treatment. Within one month, most all of the nails were growing out with normal nails below the fungal infected outer edge. By two to three months, all of the toe nails had been eliminated from their fungus. The other patients had involvements of lesser degree, but all patients had eliminated their toe nail fungus in a similar time frame.

### Example 15

A 66 year old white male had chronic, severe onychomycosis involving fungal infections of all of his toe nails and was being followed by a podiatrist. After trying several known potential remedies over months to a few years, the podiatrist told this patient that there was nothing else topically to help his problem. He asked to schedule him for removal of all of his toenails so he could remove the difficult to heal infected toenails and clear the growth beds that were not responding to his treatment. He delayed the proposed surgery and started applying the disclosed composition daily to his nails after showering. Before starting the treatment, the podiatrist cleaned the extra debris from the infection and cut the nails close. Within a few weeks, the toenails began to heal and growing out healthy nails from the nail beds. When all of the infected toenail and base had been cleared, he had normal looking nails and no longer needed the surgery the podiatrist wanted to perform.

### Example 16

A 69 year old white male had developed chronic onychomycosis, or toe nail fungus, involving most of the toe nails on both feet, but with different degrees of infection. The disclosed composition was applied once a day on all of the nails after shower or bath. The nails were to be kept well clipped and the disclosed composition applied daily. Over a few weeks, the mildly infected nails cleared of their fungus, but the more heavily infected nails took a few more weeks to clear. But, at the end, all of the nails recovered from their fungal infection.

Table 9 lists the composition formulation for treatment of toe nail fungus.

TABLE 9
Toe Nail Fungus Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Acai Berry | 10 | ml | AF (Artic Fish) Collagen (AFC) | 40 | g |
| Anethole Anise Camphor | 5 | ml | Agave nectar (AGV) | 10 | ml |
| Argan Nut Oil (AGN) | 5 | ml | Alanine (ALA) | 5 | g |
| Baobab Oil (BAB) | 5 | ml | Aloe Vera Leaf Gel (AVLG) | 275 | g |
| Bees Wax (BW) | 40 | g | Arginine (ARG) | 5 | g |
| Benzoin (BZN) | 4 | ml | Distilled water | 500 | ml |
| Benzyl Alcohol (BA) | 50 | ml | Ferulic Acid (FRA) | 10 | g |
| Cabreuva Oil | 5 | ml | Glutamine (GLU) | 5 | g |
| Calendula Oil (CDA) | 5 | ml | Glycine (GLY) | 5 | g |
| Camellia Oil (CMA) | 10 | ml | Histidine (HST) | 5 | g |
| Capaiba Oil (CPO) | 10 | ml | Hydroxyproline (HDP) | 5 | g |
| Caprylic acid (CCT) | 10 | ml | Isoleucine (ISL) | 5 | g |
| Carvacrol (CVC) | 5 | ml | Lecithin (LCT) | 25 | g |
| Cetyl Alcohol (CTA) | 40 | g | Leucine (LUC) | 5 | g |
| Chamomile German Oil (CGB) | 10 | ml | Linoleic Acid (LOA) | 5 | ml |
| Ghana Shea Butter (GSB) | 275 | g | Lysine (LYS) | 5 | g |
| Glycerol (GLY) | 10 | ml | Methyl Sulfonyl Methane (MSM) | 25 | gm |
| Kanuka Oil (KNO) | 2.5 | ml | Niacinamide (NCM) | 2.5 | g |
| Limonene Oil (LMN) | 40 | ml | Oleic Acid (OLA) | 5 | ml |
| Litsea Cubeba Oil (LCO) | 5 | ml | Proline (PRL) | 5 | g |
| Myrrh | 5 | ml | Protocatechuic Acid (PCT) | 10 | g |
| Niaouli Oil | 5 | ml | Sericin | 50 | g |
| Black Cumin Seed Oil (BCS) | 50 | ml | Sodium Polyacrylate | 20 | g |
| Panthenol (PNT) | 10 | ml | Vitamin A (VTA) | 2.5 | g |
| Peppermint | 5 | ml | Vitamin B3 (VTB3) or Niacin | 12.5 | g |
| Sea Buckthorn Oil (SBO) | 5 | ml | Vitamin C (VTC) | 2.5 | g |
| Terpineol | 5 | ml | Vitamin E (VTE) | 2.5 | g |
| Thyme Red, White | 5 | ml | | | |
| Winter Savory | 5 | ml | | | |

### Aphthous Ulcers

### Example 17

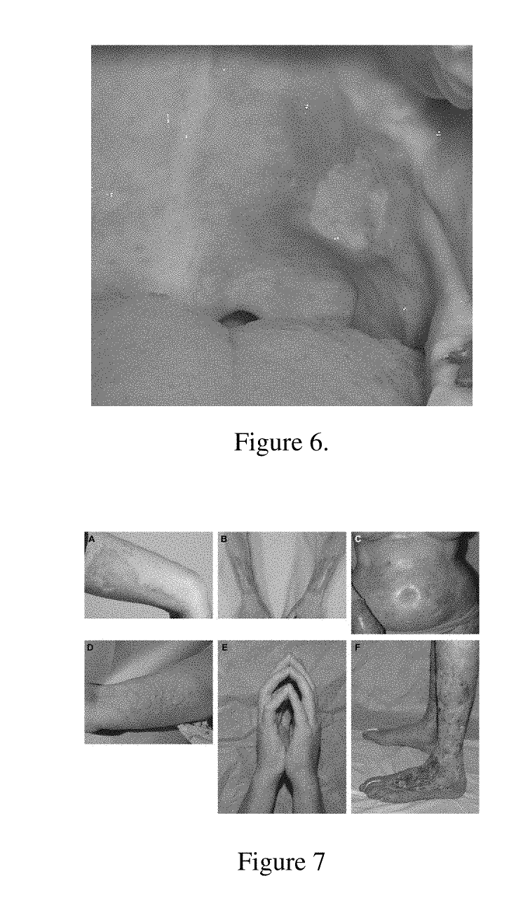

Aphthous ulcers or stomatitis are characterized by repeated, benign and non-contagious oral cavity ulcerations that are self-limited to usually 7-10 days, as shown in FIG. 6. These are painful and sensitive to increased pain by topical exposure to compositions with excessive pH, spices, and increased temperature. Episodes usually occur 3-6 times a year for several years around the time of adolescence. There is no known cause, but are thought to be multi-factorial.

While the ulceration triggers are not identified, the ulcers develop due to immune cell reactions from T-cells, mast cells, and macrophages to unknown triggers with the emphasis on T-cell involvement. Forty percent of persons getting these lesions have a family history of involvement. Some episodes seem to clearly be responding to acute, excessive stress in those who get these ulcers, but most do not. The natural duration of 7-10 days remains fairly uniform, even in spite of several different types of proposed treatments.

The disclosed composition has demonstrated the ability to reduce the duration of these ulcers to 2-4 days while relieving the pain. However, it does not appear to prevent the regular occurrence of these ulcers. Approximately 10 patients have used this formulation and uniformly experience a shorter duration of time and a reduction of pain.

### Example 18

A 65 year old white male, was having occasional episodes of aphthous ulcers of the mouth that were typical in severity and duration with pain lasting 7 to 10 days and final clearing from 2 to 2.5 weeks. These aphthous ulcers were occurring every three to four months without an obvious cause. The disclosed composition was applied at the start of an episode. The disclosed composition was designed to be thick enough to stay within the bed of the ulcer with application 2 to 4 times a day. From starting the use of the disclosed composition on the first day of the lesion appearing, and treating it 3 times a day after meals and again at bed time, the ulcer lost its pain in 2-3 days and disappeared in 4-5 days. Due to benzocaine being used as a product ingredient, the ulcer had a significant decrease in pain during the treatment phase and prior to its healing.

Table 10 lists the composition formulation for treatment of aphthous ulcers of the mouth.

TABLE 10
Aphthous Ulcers of the Mouth Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Acai Berry | 10 | ml | AF (Artic Fish) Collagen (AFC) | 40 | g |
| Anethole Anise Camphor | 5 | ml | Agave nectar (AGV) | 10 | ml |
| Argan Nut Oil (AGN) | 5 | ml | Alanine (ALA) | 5 | g |
| Baobab Oil (BAB) | 5 | ml | Aloe Vera Leaf Gel (AVLG) | 275 | g |
| Bees Wax (BW) | 40 | g | Arginine (ARG) | 5 | g |
| Benzoin (BZN) | 4 | ml | Benzocaine | 3 | g |
| Benzyl Alcohol (BA) | 50 | ml | Distilled water | 500 | ml |
| Cabreuva Oil | 5 | ml | Ferulic Acid (FRA) | 10 | g |
| Caffeic acid | 5 | g | Glutamine (GLU) | 5 | g |
| Calendula Oil (CDA) | 5 | ml | Glycine (GLY) | 5 | g |
| Capaiba Oil (CPO) | 10 | ml | Histidine (HST) | 5 | g |
| Caprylic acid (CCT) | 10 | ml | Hydroxyproline (HDP) | 5 | g |
| Carvacrol (CVC) | 5 | ml | Isoleucine (ISL) | 5 | g |
| Caryophyllene (CAP) | 10 | ml | Lecithin (LCT) | 25 | g |
| Cedarwood essential Oil | 2.5 | nl | Leucine (LUC) | 5 | g |
| Cetyl Alcohol (CTA) | 40 | g | Linoleic Acid (LOA) | 5 | ml |
| Chamomile German Oil (CGB) | 10 | ml | Lysine (LYS) | 5 | g |
| Ghana Shea Butter (GSB) | 275 | g | Methyl Sulfonyl Methane (MSM) | 25 | g |
| Glycerol (GLY) | 10 | ml | Niacinamide (NCM) | 2.5 | g |
| Kanuka Oil (KNO) | 2.5 | ml | Oleic Acid (OLA) | 5 | ml |
| Limonene Oil (LMN) | 40 | ml | Proline (PRL) | 5 | g |
| Litsea Cubeba Oil (LCO) | 5 | ml | Protocatechuic Acid (PCT) | 10 | g |
| Niaouli Oil | 5 | ml | Sericin | 50 | g |
| Black Cumin Seed Oil (BCS) | 50 | ml | Sodium Polyacrylate | 20 | g |
| Panthenol (PNT) | 10 | ml | Vitamin A (VTA) | 2.5 | g |
| Peppermint | 5 | ml | Vitamin B3 (VTB3) or Niacin | 12.5 | g |
| Sea Buckthorn Oil (SBO) | 5 | ml | Vitamin C (VTC) | 2.5 | g |
| Thyme Red, White | 5 | ml | Vitamin E (VTE) | 2.5 | g |
| Winter Savory | 5 | ml | | | |

### Cutaneous Involvement of Scleroderma

### Example 19

Scleroderma is a systemic disease that results in progressive fibrosis of organs starting with skin lesions that can then progress to involving major organs, such as, the heart, lungs, intestines and kidneys. The pathophysiology for the skin appears to involve the laying down in a random orientation of collagen much like scar formation diffusively in the subcutaneous tissues. This leads to joint immobilization starting in the fingers and spreading into the wrists and elbows as well as in the toes and ankle (FIG. 7). Its increased pressure beneath the skin also reduces blood flow causing extensive Raynaud's phenomenon, or skin blanching episodes on the digits that readily leads to poorly healing ulcerations and potential digit loss.

A white female, 52 years old developed Scleroderma with cutaneous manifestations that resulted in contractures of all the joints of the fingers on both hands, both wrists, and early limitations of both elbows. Fibrosis of the fingers only left about 5-10% motion in all fingers. The wrist joints were about 50% inhibited with elbows about 30% inhibited, all due to the subcutaneous fibrosis. She had evidence of some areas of collagen formation beneath the skin in both upper extremities. The patient's primary complaint was that her skin was giving her so many symptoms on a 24 hour basis; she was treating subcutaneous discomfort in both arms with over the counter skin moisturizers every two hours without much relief, including at night. She tested the disclosed composition on the skin of both arms and found symptomatic relief. Instead of every two hours, she found by applying this product only every 12 hours, she got improved relief of her previous cutaneous symptoms. In addition, she had developed significant Raynaud's Phenomenon on fingers on both hands with early joint ulcerations noted on several fingers. With some early systemic symptoms, she was treated with an oral drug for Scleroderma for several months but systemic disease progression continued. She underwent bone marrow ablation and received a bone marrow transplant and is currently under remission. The disclosed composition was discontinued when her disease progression began to advance to systemic symptoms.

Table 11 lists the composition formulation for treatment of cutaneous scleroderma.

TABLE 11
Cutaneous Scleroderma Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Acai Berry | 10 | ml | AF (Artic Fish) Collagen (AFC) | 40 | g |
| Argan Nut Oil (AGN) | 5 | ml | Agave nectar (AGV) | 10 | ml |
| Baobab Oil (BAB) | 5 | ml | Alanine (ALA) | 5 | g |
| Bees Wax (BW) | 40 | g | Aloe Vera Leaf Gel (AVLG) | 275 | g |
| Benzoin (BZN) | 4 | ml | Arginine (ARG) | 5 | g |
| Benzyl Alcohol (BA) | 50 | ml | Distilled water | 500 | ml |
| Calendula Oil (CDA) | 5 | ml | Ferulic Acid (FRA) | 10 | g |
| Camellia Oil (CMA) | 10 | ml | Glutamine (GLU) | 5 | g |
| Capaiba Oil (CPO) | 10 | ml | Glycine (GLY) | 5 | g |
| Caprylic, Capric Triglyceride (CCT) | 10 | ml | Histidine (HST) | 5 | g |
| Carvacrol (CVC) | 5 | ml | Hydroxyproline (HDP) | 5 | g |
| Caryophyllene (CAP) | 10 | ml | Isoleucine (ISL) | 5 | g |
| Cetyl Alcohol (CTA) | 40 | g | Lecithin (LCT) | 25 | g |
| Chamomile German Oil (CGB) | 10 | ml | Leucine (LUC) | 5 | g |
| Elemi Oil (EMO) | 5 | ml | Linoleic Acid (LOA) | 5 | ml |
| Geranium Oil (GER) | 2.5 | ml | Lysine (LYS) | 5 | g |
| Ghana Shea Butter (GSB) | 275 | g | Methyl Sulfonyl Methane (MSM) | 25 | g |
| Glycerol (GLY) | 10 | ml | Niacinamide (NCM) | 2.5 | g |
| Ho Wood Oil (HWO) | 2.5 | ml | Oleic Acid (OLA) | 5 | ml |
| Jasmine Oil (JAS) | 2.5 | ml | Proline (PRL) | 5 | g |
| Kanuka Oil (KNO) | 2.5 | ml | Protocatechuic Acid (PCT) | 10 | g |
| Lavandin Oil (LVO) | 10 | ml | Sericin | 50 | g |
| Limonene Oil (LMN) | 40 | ml | Sodium Polyacrylate | 20 | g |
| Litsea Cubeba Oil (LCO) | 5 | ml | Vitamin A (VTA) | 2.5 | g |
| Marula Oil (MRL) | 2.5 | ml | Vitamin B3 (VTB3) or Niacin | 12.5 | g |
| Nerolina Oil (NRL) | 2.5 | ml | Vitamin C (VTC) | 2.5 | g |
| Black Cumin Seed Oil (BCS) | 50 | ml | Vitamin E (VTE) | 2.5 | g |
| Palo Santo | 2.5 | ml | | | |
| Panthenol (PNT) | 10 | ml | | | |
| Rose Hip Seed Oil | 10 | ml | | | |
| Rosemary Oil (RMO) | 5 | ml | | | |
| Sachi Inchi oil | 5 | ml | | | |
| Sea Buckthorn Oil (SBO) | 5 | ml | | | |
| Tamanu Oil (TMU) | 5 | ml | | | |

### Raynaud's (or Renaud's) Phenomenon Preparation

### Example 20

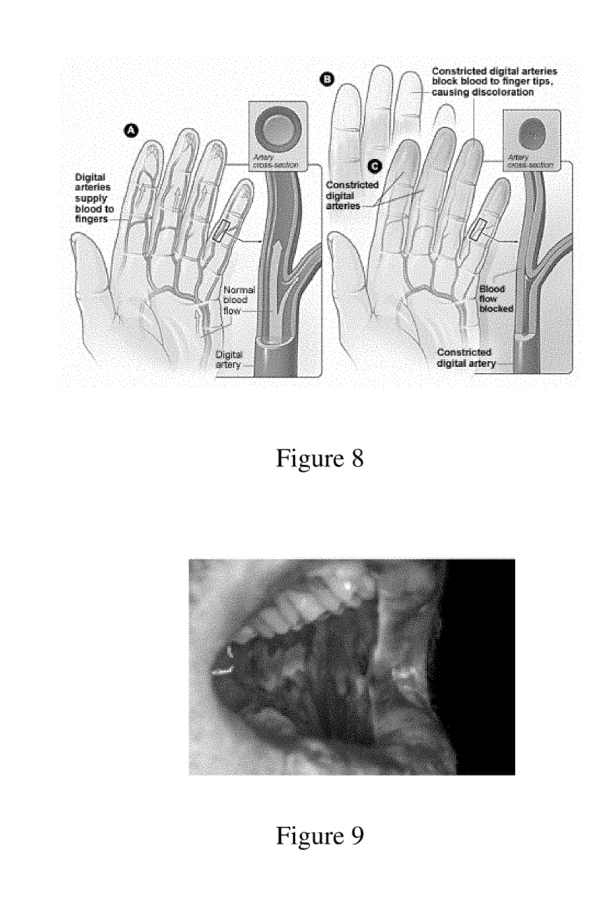

Raynaud's Phenomenon or Syndrome is a medical condition that involves episodic spasm of the arteries that markedly reduces blood flow to the hands and/or feet that can eventually lead to poorly healing ulcers and to gangrene with loss of digits (FIG. 8). The episodes affecting the hands or feet initially turn white and later blue, often with associated numbness or pain. As the spasm ends with blood flow returning, the affected parts turn red and are typically painful. These episodes usually only last a few minutes, but can last for hours. There are two major types of Raynaud's Phenomenon that includes 4% of the population: primary without a known cause and secondary which occurs related to other primary conditions. The secondary conditions that can cause these episodes include immune disorders, such as, lupus and scleroderma, former trauma including frost bite, smoking, thyroid problems, and certain medications, such as, birth control pills. The existing primary treatment is to eliminate known stimulants, such as, smoking and to avoid exposing the affected extremities to cold temperatures. If these are not effective, then calcium channel blockers or a vasodilator can be tried. There currently are no other known remedies.

The patient that presented with scleroderma also suffered from Raynaud's phenomenon in both hands with sufficient frequency and intensity that multiple ulcers formed on the dorsum of several digits. The disclosed composition for treatment of Raynaud's Phenomenon was applied to the hands, between episodes of Raynaud's attacks, of the patient with scleroderma and improved the color of the fingers which also were found by palpation to be warmer.

Table 12 lists the composition formulation for treatment of Raynaud's Phenomenon.

TABLE 12
Raynaud's Phenomenon Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Acai Berry | 10 | ml | AF (Artic Fish) Collagen (AFC) | 40 | g |
| Argan Nut Oil (AGN) | 5 | ml | Agave nectar (AGV) | 10 | ml |
| Baobab Oil (BAB) | 5 | ml | Alanine (ALA) | 5 | g |
| Bees Wax (BW) | 40 | g | Aloe Vera Leaf Gel (AVLG) | 275 | g |
| Benzoin (BZN) | 4 | ml | Arginine (ARG) | 5 | g |
| Benzyl Alcohol (BA) | 50 | ml | Distilled water | 500 | ml |
| Caffeic acid | 5 | g | Ferulic Acid (FRA) | 10 | g |
| Calendula Oil (CDA) | 5 | ml | Glutamine (GLU) | 5 | g |
| Camellia Oil (CMA) | 10 | ml | Glycine (GLY) | 5 | g |
| Capaiba Oil (CPO) | 10 | ml | Histidine (HST) | 5 | g |
| Caprylic, Capric Triglyceride (CCT) | 10 | ml | Hydroxyproline (HDP) | 5 | g |
| Carvacrol (CVC) | 5 | ml | Isoleucine (ISL) | 5 | g |
| Cetyl Alcohol (CTA) | 40 | g | Lecithin (LCT) | 25 | g |
| Chamomile German Oil (CGB) | 10 | ml | Leucine (LUC) | 5 | g |
| Ghana Shea Butter (GSB) | 275 | g | Linoleic Acid (LOA) | 5 | ml |
| Glycerol (GLY) | 10 | ml | Lysine (LYS) | 5 | g |
| Kanuka Oil (KNO) | 2.5 | ml | Methyl Sulfonyl Methane (MSM) | 25 | g |
| Limonene Oil (LMN) | 40 | ml | Niacinamide (NCM) | 2.5 | g |
| Litsea Cubeba Oil (LCO) | 5 | ml | Oleic Acid (OLA) | 5 | ml |
| Black Cumin Seed Oil (BCS) | 50 | ml | Proline (PRL) | 5 | g |
| Panthenol (PNT) | 10 | ml | Protocatechuic Acid (PCT) | 10 | g |
| Terpineol | 5 | ml | Sericin | 50 | g |
| | | | Sodium Polyacrylate | 20 | g |
| | | | Vitamin A (VTA) | 2.5 | g |
| | | | Vitamin B3 (VTB3) or Niacin | 12.5 | g |
| | | | Vitamin C (VTC) | 2.5 | g |
| | | | Vitamin E (VTE) | 2.5 | g |

### Chemotherapy/Radiation Therapy Induced Oral Mucosal Lesions

### Example 21

A common problem with both systemic irradiation and chemotherapy is the development of multiple induced oral mucosal ulcers scattered throughout the mouth to the 40-60% patient incidence level (FIG. 9). These are very painful and have no known treatment at the moment. The pathophysiology of these ulcers relates to the fact that the oral mucosal lining cells normally have a rapid turnover and replacement time. Whenever chemotherapy and/or radiation therapy in the body occurs, the turnover replication rate of these oral mucosal lining cells reduces significantly. This means these cells cannot replicate sufficiently fast enough to replace their normal losses resulting in the formation of a mucosa with major gaps in their surface cell coverage.

Currently there is no treatment for this painful consequence of the tumor treatment. The significant factor one has to consider in developing a replacement is that the increased cell replication agent cannot interfere with the treatment of the tumor. This means that the treatment has to remain local within the mouth and cannot be absorbed into the body that is being treated for systemic cancers.

Table 13 lists the composition formulation for treatment of chemotherapy/radiation therapy induced oral mucosal lesions.

TABLE 13
Chemotherapy/Radiation Therapy Induced Oral Mucosal Lesions Composition Formulation

| Hydrophobic Components | amount | | Hydrophilic Components | amount | |
|---|---|---|---|---|---|
| α-Pinene (APN) | 60 | ml | 1-Tetradecanol | 2.5 | g |
| Acai Berry | 10 | ml | AF (Artic Fish) Collagen (AFC) | 40 | g |
| Anethole Anise Camphor | 5 | ml | Agave nectar (AGV) | 10 | ml |
| Argan Nut Oil (AGN) | 5 | ml | Alanine (ALA) | 5 | g |
| Baobab Oil (BAB) | 5 | ml | Aloe Vera Leaf Gel (AVLG) | 275 | g |
| Bees Wax (BW) | 40 | g | Arginine (ARG) | 5 | g |
| Benzoin (BZN) | 4 | ml | Distilled water | 500 | ml |
| Benzyl Alcohol (BA) | 50 | ml | Ferulic Acid (FRA) | 10 | g |
| Cabreuva Oil | 5 | ml | Glutamine (GLU) | 5 | g |
| Calendula Oil (CDA) | 5 | ml | Glycine (GLY) | 5 | g |
| Camellia Oil (CMA) | 10 | ml | Histidine (HST) | 5 | g |
| Capaiba Oil (CPO) | 10 | ml | Hydroxyproline (HDP) | 5 | g |
| Caprylic, Capric Triglyceride (CCT) | 10 | ml | Isoleucine (ISL) | 5 | g |
| Carvacrol (CVC) | 5 | ml | Lecithin (LCT) | 25 | g |
| Caryophyllene (CAP) | 10 | ml | Leucine (LUC) | 5 | g |
| Cedarwood essential Oil | 2.5 | ml | Linoleic Acid (LOA) | 5 | ml |
| Cetyl Alcohol (CTA) | 40 | g | Lysine (LYS) | 5 | g |
| Chamomile German Oil (CGB) | 10 | ml | Methyl Sulfonyl Methane (MSM) | 25 | g |
| Ghana Shea Butter (GSB) | 275 | g | Niacinamide (NCM) | 2.5 | g |
| Glycerol (GLY) | 10 | ml | Oleic Acid (OLA) | 5 | ml |
| Kaempferol | 5 | g | Proline (PRL) | 5 | g |
| Kanuka Oil (KNO) | 2.5 | ml | Protocatechuic Acid (PCT) | 10 | g |
| Limonene Oil (LMN) | 40 | ml | Sericin | 50 | g |
| Litsea Cubeba Oil (LCO) | 5 | ml | Sodium Polyacrylate | 20 | g |
| Niaouli Oil | 5 | ml | Vitamin A (VTA) | 2.5 | g |
| Black Cumin Seed Oil (BCS) | 50 | ml | Vitamin B3 (VTB3) or Niacin | 12.5 | g |
| Panthenol (PNT) | 10 | ml | Vitamin C (VTC) | 2.5 | g |
| Peppermint | 5 | ml | Vitamin E (VTE) | 2.5 | g |
| Sea Buckthorn Oil (SBO) | 5 | ml | | | |
| Thyme Red, White | 5 | ml | | | |
| Winter Savory | 5 | ml | | | |

### Methods of Making Compositions

### Example 22

The methods involved in producing these compositions are generally similar, but have different components for each composition. There are hydrophobic components and hydrophilic components that must be initially mixed separately under different temperature controls and then combined together properly. As many of these components are not liquid at room temperature, heating to phase change so they all become liquids is the first requirement. The first temperature level for the first mixture is set at 40° C. to 50° C., preferably 45° C., with a 300 to 500 rotations per minute (RPM), preferably 400 RPM, propeller mixing speed. All of the components that are a liquid or become a liquid at that temperature are mixed together in one of two vessels: a hydrophobic vessel and a hydrophilic vessel. The remaining unmixed components require higher temperatures to become liquid. Thus, the temperatures of the mixtures are increased to 60° C. to 80° C., preferably 60° C., and the speed of rotation is increased to 700 to 900 RPM, preferably 800 RPM. The remaining hydrophobic group components are added to its vessel and the hydrophilic group components are added to its vessel. After sufficient time of 1 to 8 hours, preferably more than 8 hours, for mixing and completion of reaction in each of the two vessels, the mixed hydrophilic group of components is then poured into the hydrophobic group vessel, maintaining the 700 to 900 RPM, preferably 800 RPM and the 60° C. to 80° C., preferably 60° C., temperature for another 10-40 minutes, preferably 20 minutes, of reaction time. At this point, the mixture is slowly cooled to room temperature over several hours of 2 to 10 hours while reducing the mixing speed to 500-700 RPM, preferably 600 RPM, continuing to insulate the mixing container. This lets the mixture cool to ambient room temperature of 25° C. to 37° C., preferably 25° C., over a slow period time while continuously being thoroughly mixed. When cooled to room temperature, the propeller is stopped and this final mixing vessel is removed to a surface top. This final composition will remain semisolid until it is returned to partial liquid at 37° C. by design to melt onto the skin when applied. Filling individual pumps (15 ml to 50 ml) is the final step prior to storage and then for delivery of this composition to the purchaser on a regular basis.

1. A composition comprising α-pinene, ghana shea butter and methylsulfonylmethane.

2. The composition of claim 1 further comprising glycerol, black cumin seed, aloe vera gel, collagen and lecithin.

3. The composition of claim 2 further comprising at least one from the group consisting of functional oil, alcohol, anti-inflammatory, anti-oxidant, anti-microbial, floral, thickener, wax, fragrance, fatty acid, analgesic, amino acid, carbohydrate, vitamin, solvent, gel, and moisturizer.

4. The composition of claim 3 wherein the functional oil is selected from the group consisting of apricot kernal oil, argan nut oil, baobab oil, calendula oil, camellia oil, caprylic, capric triglyceride, caryophyllene, elemi oil, kanuka oil, litsea cubebe oil, marula oil, palmarosa oil, palo santo, rose hip seed oil, sachi inchi oil, sea buckthorn oil and tamanu oil.

5. The composition of claim 3 wherein the alcohol is selected from the group consisting of benzyl alcohol, cetyl alcohol and panthenol.

6. The composition of claim 3 wherein the anti-inflammatory is selected from the group consisting of acai berry, chamomile german oil, capaiba oil, myrrh, rose hip seed oil, oleic acid and linoleic acid.

7. The composition of claim 3 wherein the anti-oxidant is selected from the group consisting of kaempferol, caffeic acid and protocatechuic acid.

8. The composition of claim 3 wherein the anti-microbial is selected from the group consisting of anethole anise camphor, cabreuva oil, carvacrol, niaouli oil, thyme and winter savory.

9. The composition of claim 3 wherein the floral is rosemary oil.

10. The composition of claim 3 wherein the thickener is benzoin.

11. The composition of claim 3 wherein the wax is bees wax.

12. The composition of claim 3 wherein the fragrance is selected from the group consisting of carvone oil, geranium oil, ho wood oil, jasmine oil, lavodin oil, limonene oil, nerolina oil, peppermint, terpineol and vetiver.

13. The composition of claim 3 wherein the fatty acid is ferulic acid.

14. The composition of claim 3 wherein the analgesic is benzocaine.

15. The composition of claim 3 wherein the amino acid is selected from the group consisting of alanine, arginine, glutamine, glycine, histidine, hydroxyproline, isoleucine, leucine, lysine and proline.

16. The composition of claim 3 wherein the carbohydrate is agave nectar.

17. The composition of claim 3 wherein the vitamin is selected from the group consisting of Vitamin A, Vitamin B3, Niacin, Vitamin C, Vitamin E, Niacinamide.

18. The composition of claim 3 wherein the gel is sericin.

19. The composition of claim 3 wherein the moisturizer is selected from the group consisting of sodium polyacrylate and 1-tetradecanol.

20. A method of combining hydrophobic components and hydrophilic components with high dosage of active ingredients, comprising the steps of:
a) placing hydrophobic components that are a liquid or become a liquid at 40° C. to 50° C. in a first vessel and placing hydrophilic components that are a liquid or become a liquid at 40° C. to 50° C. in a second vessel;
b) inserting into each of the first vessel and the second vessel a propeller mixer rotating at 300 to 500 rotations per minute (RPM) propeller mixing speed and heating each of the first vessel and the second vessel to 40° C. to 50° C.;
c) after sufficient mixing at 40° C. to 50° C., placing hydrophobic components that are not liquid at 40° C. to 50° C. in the first vessel and placing hydrophilic components that are not liquid at 40° C. to 50° C. in the second vessel, and increasing rotation of each propeller mixer to 700 to 900 RPM and increasing the temperature of the first vessel and the second vessel to 60° C. to 80° C.;
d) after sufficient time for mixing and completion of reaction in the first vessel and the second vessel, pouring the contents of the second vessel into the first vessel and combining the contents of both vessels, and maintaining the rotation of the propeller mixer at 700 to 900 RPM and temperature at 60° C. to 80° C. for an additional 10-40 minutes of reaction time;
e) adjusting pH to 7.5 with NaOH;
f) cooling the mixed contents in the first vessel slowly over several hours to ambient temperature by insulating the first vessel and reducing the propeller mixer to 500-700 RPM;
g) stopping the propeller mixer after the contents in the first vessel reaches ambient temperature;
h) filling mixed content into a container.
